(19) United States
(12) Patent Application Publication
(10) Pub. No.: US 2008/0008685 A1
(43) Pub. Date: Jan. 10, 2008

(54) Viral vectors

(76) Inventor: Noriyuki Kasahara, Los Angeles, CA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/805,411

(22) Filed: May 23, 2007

Related U.S. Application Data
(63) Continuation of application No. PCT/US05/42774, filed on Nov. 23, 2005.
(60) Provisional application No. 60/630,963, filed on Nov. 24, 2004.

(51) Int. Cl. A61K 48/00; A61K 31/711; A61P 25/16; A61P 3/10; A61P 31/12; A61P 35/00; C12N 15/64; C12N 15/86

(52) U.S. Cl. 424/93.2; 435/320.1; 435/91.4; 514/44

(57) ABSTRACT

The present invention provides a plasmid encoding a replication-competent virus for use in therapy more particularly for use in the treatment of a cell proliferative disease, an immunological disease, a neuronal disorder, an acquired infection and inflammation as well as formulations comprising such plasmids together with a transfection agent.

### CROSS-REFERENCE TO RELATED APPLICATION(S)

This application is a continuation of International Patent Application No. PCT/US2005/042774, filed on Nov. 23, 2005, which claims priority to German Application No. 60/630,963, filed on Nov. 24, 2004, the contents of which are hereby incorporated in their entirety by reference herein.

This application also claims priority of U.S. Application No. 60/630,963 filed Nov. 24, 2004, the disclosure of which is incorporated herein by reference.

### FIELD OF THE INVENTION

The present invention relates to the field of delivery of replication competent viruses in vivo for therapeutic purposes and specifically to novel ways in which such viruses may be introduced into a patient or host.

### BACKGROUND OF THE INVENTION

Viruses are now widely used therapeutic agents in the fight against disease, both in non-human animals and humans. Particularly, viruses are utilised as vectors for gene therapy. Alternatively, cytolytic viruses have been used to target and kill cancer or unwanted proliferating cells, such therapy is also known as “virotherapy”. Thus, the direct use of viruses in medical treatments is a widely growing area, and new techniques and uses involving viruses in treatment and therapy are being developed.

Viruses are highly evolved biological entities that efficiently gain access to their host cells and exploit the cellular machinery of the cell to facilitate their replication. As such, they are heralded as ideal gene therapy vectors since foreign/heterologous genes or coding sequences may be inserted into the viral genome and infection thus allows the foreign gene to be delivered to the nucleus of the host cell. Gene therapy was first conceived in order to treat genetic diseases where the defect lay in a hereditary single-gene defect, for example severe combined immunodeficiency disease (SCID). However, the scope of gene therapy is now much broader, and it is envisaged that viruses may be utilised to deliver genes for acquired diseases such as cancer, cardiovascular disease, neurodegenerative disorders, inflammation and even infectious disease.

### BRIEF SUMMARY OF THE INVENTION

The present invention thus aims to utilise a plasmid encoding a recombinant replication-competent virus in the transfection of a cell in vivo. Once a cell has been successfully transfected with a plasmid, expression of the recombinant replication—competent virus will occur, and this will in due course lead to the virus being released from the cell and being able to transfect target cells. Thus, from a single initial plasmid transfection event, multiple target cells may be transfected. It has been identified here for the first time that plasmid may be utilised in such a way. As mentioned previously, prior gene therapy and viral therapy methods have focused upon delivery of the virus itself, and use of the plasmid encoding the virus is elegantly simple, and overcomes some of the problems hereinbefore discussed.

Accordingly, in one aspect the present invention provides a plasmid encoding a replication-competent virus for use in therapy.

In another aspect, replication-competent retrovirus comprises:
a retroviral GAG coding sequence;
a retroviral POL coding sequence;
a retroviral ENV coding sequence;
retroviral Long Terminal Repeat (LTR) sequences;
and optionally one or more of the following elements;
a heterologous coding sequence operably linked to a regulatory nucleic acid sequence; and
one or more targeting sequences for cell- or tissue-specific targeting of the retrovirus.

In another aspect, the viral genome incorporates a therapeutic gene or coding sequence suitable for the treatment of a condition which the patient is suffering from Such conditions include cell proliferative diseases, immunological disease, neuronal disorders, an acquired infection or inflammation. Additionally, the condition can be a cancer, severe combined immunodeficiency disease, Parkinson's disease, Hepatitis C infection or diabetes.

### BRIEF DESCRIPTION OF THE DRAWINGS

FIG. 1 provides a schematic diagram of the structure of a nucleic acid molecule encoding a replication-competent MoMLV retrovirus of the invention.

FIG. 2 provides a plasmid encoding a replication-competent retrovirus.

FIG. 3A depicts the general structure of nucleic acid encoding a replication-competent retrovirus.

FIG. 3B provides particular plasmid vectors, indicating the identity of the transgene insert and the sequence at both ends of the transgene insert.

FIG. 4 provides the replication curves of plasmid pACE-GFP-derived retrovirus vectors in the WiDr human colorectal cancer cell line and the CT26.WT murine colorectal cancer cell line, respectively, after inoculation at different doses.

FIG. 5 depicts representative composite images after optical imaging of GFP fluorescence from livers isolated 48 hours after hydrodynamic injection of plasmid pACE-GFP (Example 6).

FIG. 6 provides representative composite images after optical imaging of GFP fluorescence from livers isolated on Day 21 (middle panel) and Day 28 (right panel) as described in Example 6.

FIG. 7 provides the results of fluorescence-activated cell sorter (FACS) analysis of dispersed tumor cells harvested immediately after dissection at serial time points during pACE-GFP-derived virus nanovector replication in vivo.

FIG. 8 provides the results from PCR analysis of pACE-GFP-derived replicating virus integration of the GFP transgene.

### DETAILED DESCRIPTION

There are currently five main classes of clinically applicable viral vectors that are derived from oncoretroviruses, lentivirus, adenovirus, adeno-associated—virus (AAVs) and herpes-simplex-1 viruses (HSV-1s). Each class of vector is characterised by a set of different properties that make it suitable for use in certain applications, and unsuitable for others.

The five main classes of viral vector can be categorized in two groups according to whether their genomes integrate into host DNA (oncoretroviruses and lentiviruses) or persist in the cell nucleus predominantly as extrachromosomal episomes (AAVs, adenoviruses and herpes viruses). This distinction is an important determinant of the suitability of each vector for particular applications; non-integrating vectors can, under certain circumstances, mediate persistent transgene expression in non-proliferating cells, but integrating vectors are, at present, the tools of choice if stable genetic alteration needs to be maintained in dividing cells.

Oncoretrovirus vectors were the first class of viral vector to be developed and have, so far, been the most widely used in clinical trials. They have traditionally been the vectors of choice for the ex vivo transduction of stem cells. However, most work has focused on the development of lentivirus vectors, which can naturally penetrate an intact nuclear membrane and transduce non-dividing cells. Lentivirus vectors will probably be important vector systems in the future treatment of a wide range of diseases. They have proven to be effective tools for gene delivery to the central nervous system, generating long-term gene expression in the absence of inflammation.

With regard to “virotherapy” techniques, wherein the virus is utilised for its cytolytic ability to destroy proliferating cells, such as cancer cells, it is not necessary for the virus to be carrying any foreign coding sequences or deliver any sequences to the target cell. It may be highly desirable in such strategies to use viruses that are targeted only to dividing cells, such that only the proliferating cells are destroyed. It may be possible in such cases also to insert a nucleic acid sequence into the viral vector that allows the viruses to be destroyed by the addition of a drug to the system, allowing for a further level of control. Lytic viruses include adenovirus.

Vector tropism (i.e. host cell targeting), the duration of transgene expression within the target cell and vector immunogenicity are other factors that influence the choice of a vector for specific therapeutic applications. Adenovirus vectors are, arguably, the most efficient class of vector in terms of delivering their nucleic acid sequences to the cell nucleus, and direct injection of adenovirus vectors can efficiently transduce most tissues.

Recombinant AAV vectors (rAAVs) are one of the most promising vector systems for safe long-term gene transfer and expression in non-proliferating tissues. AAV is unique among viruses that are being developed for gene therapy in that the wild-type virus has never been shown to cause human disease. The small size and simplicity of the vector particle makes it possible to administer high doses of vector systemically without eliciting acute inflammatory responses or toxic side effects.

The space available in the vector genome for the incorporation of foreign DNA is another criterion that influences the choice of vector for specific therapeutic applications.

Gene therapy vectors based on simple retroviruses, such as the Moloney Leukemia Virus (MoMLV), are often selected because they efficiently integrate into the genome of the target cell. Integration is thought to be a prerequisite for long-term expression of the transduced gene.

In the early steps of infection, retroviruses deliver their nucleoprotein core into the cytoplasm of the target cell. Here, reverse transcription of the viral genome takes place while the core matures into a prointegration complex. The complex must reach the nucleus to achieve integration of the viral DNA into the host cell chromosomes. For simple retroviruses (oncoretroviruses), this step requires the dissolution of the nuclear membrane during cell division, most likely because the bulky size of the protein/nucleic acid complex prevents its passive diffusion through the nuclear pores because there are no clear localization signals to facilitate active transport into the nucleus.

Currently most retroviral vectors used for human gene therapy are replication-defective and must be produced in packaging cells, which contain integrated wild type virus genome sequences and thus provide all of the structural elements necessary to assemble viruses, but cannot encapsidate their own wild type virus genomes due to a deletion of the packaging signal sequence (psi).

Generally, replication-defective retroviral vectors are produced from the packaging cells at titres of the order of only 10⁶⁻⁷ colony-forming units (cfu) per ml, which is barely adequate for transduction in vivo.

However, the present invention is generally concerned with viruses that are replication competent, and seeks to deal with the inadequacies of viruses for therapy which have been produced in vitro.

In order to produce viruses for therapy that are of a sufficient clinical grade to introduce to animals, including humans, the viruses must be produced according to stringent requirements. In order to produce a virus, generally the recombinant virus is initially constructed as a plasmid comprising viral sequences, and the therapeutic gene if necessary. This plasmid is transfected into cells in vitro and the viruses produced by the transfected cells are collected and purified, should the virus for therapy be competent of self-replication. In some cases, it is desired to make the virus incapable of replication, and thus they must be produced in packaging cells. The present invention is not concerned with viruses that are incapable of self-replication.

For clinical grade virus production, expensive and technically demanding large scale mammalian cell cultures must be grown. This generally requires constant maintenance of sterile conditions, using bioreactors with large surface areas (as most mammalian cells need to adhere to a surface or they undergo apoptosis) and constant perfusion and replenishment of medium for long periods of time (since most mammalian cells divide only once every 24 hours on average, the scale up process can take a long time). Upon harvest, the viruses must be purified with gentle but inefficient methods, especially in the case of lipid-enveloped viruses such as retroviruses, which are quite fragile and often require low speed centrifugation or tangential flow filtration through a size exclusion filter to reduce volume and concentrate the virus preparation. Whilst ultracentrifugation procedures to pellet retroviruses are possible, generally much of the virus particles are destroyed upon pelleting, resulting in a significant net loss of overall yield in exchange for a somewhat more concentrated preparation in a small volume. Thus, it can be seen that the production of clinical grade viruses to use in therapy can be time-consuming, expensive, and ultimately result in low yields.

Accordingly, there is a need to overcome the problems presented by the lengthy culturing and purification steps that take place in current viral therapy methods.

The present invention seeks to overcome the problems with the production of clinical grade viruses in vitro by the complete removal of this step. The present inventors thus propose the direct utilisation of plasmids in the transfection of cells in vivo, in order to produce recombinant replication-competent viruses in vivo. Such an elegant solution has not been previously contemplated by those working in the art.

This significant shift from current practice underlies the present invention. In particular, the direct transfection of cells in vivo allows for direct viral production within the cell type, organ or tissue of choice and permits localised transfection of target cells following a low frequency transfection event. Also, if the virion produced in vivo incorporates a tropism of a target cell type, non-target cell types can be initially transfected with the plasmid and behave as producer cells. Clearly this offers great flexibility to the therapeutic methods.

The present invention thus aims to utilise a plasmid encoding a recombinant replication-competent virus in the transfection of a cell in vivo. Once a cell has been successfully transfected with a plasmid, expression of the recombinant replication—competent virus will occur, and this will in due course lead to the virus being released from the cell and being able to transfect target cells. Thus, from a single initial plasmid transfection event, multiple target cells may be transfected. It has been identified here for the first time that plasmid may be utilised in such a way. As mentioned previously, prior gene therapy and viral therapy methods have focused upon delivery of the virus itself, and use of the plasmid encoding the virus is elegantly simple, and overcomes some of the problems hereinbefore discussed.

Accordingly, in one aspect the present invention provides a plasmid encoding a replication-competent virus for use in therapy.

The term “plasmid” as used herein refers to a nucleic acid structure which is capable of existing extrachromosomally in a cell. It is thus capable of autonomous existence and constitutes a separate replicon. It may be a DNA or RNA structure, preferably a DNA structure, either in single stranded or double stranded form. Generally, plasmid molecules are circular nucleic acid molecules which contain coding sequences (genes) and regulatory sequences as described further below. However, nicked circular nucleic acid molecules and linear nucleic acid molecules are also contemplated. It is particularly preferred that the plasmid is circular double stranded DNA or a nicked version thereof (in which one of the strands is not continuously circular). The DNA may contain viral cDNA. Further, modified or unusual nucleic acid residues (e.g. incorporating inosine) may be utilized in the plasmid.

A “virus” is a non-cellular infective agent capable of reproduction in an appropriate host cell. Structurally the infective particle (virion) consists of a core of nucleic acid (DNA or RNA) surrounded by a proteinaceous capsid and, in some cases an outer envelope.

The plasmid employed in the transfection of the cell encodes a replication-competent virus. As used herein, the phrase “plasmid encoding a recombinant replication-competent virus” refers to a plasmid containing the coding sequences for a virus (either a DNA virus or an RNA virus as discussed further below) which contains all the sequences (such as coding sequences genes and promoters/enhancers) necessary for the in vivo production of a virus within the transfected cell, resulting in production of live viruses which are capable of transfecting cells in vivo upon release from the cell in which it was produced. Thus, the plasmid contains all the necessary sequences to allow for production of a virus which can transfect cells in vivo, without the need for helper viruses or packaging cells. Replication-competent viruses allow efficient transfection in vivo, since the viruses are capable of infecting target cells.

The plasmid for use in the invention comprises the viral nucleic acid sequences required for (i) the production, assembly, and release of a virion particle, (ii) the packaging of said virus sequences (referred to herein as the “viral genome”) within said particle, (iii) the cellular entry and establishment of infection by said particle, and (iv) the replication of the viral genome sequences within the infected cells.

These sequences include but are not limited to terminal repeat sequences and packaging signal sequences, as well as sequences encoding wild type or heterologous viral transcription factors, polymerases, and structural capsid and/or envelope proteins, with operably linked regulatory sequences such as wild type or heterologous promoters or enhancers. The structure of the plasmid used in the invention is further particularized below.

Any suitable virus may be employed in the invention. The virus is replication-competent as previously mentioned, and can thus multiply in situ. Suitable viruses include RNA viruses, such as retroviruses, and DNA viruses. Examples of suitable DNA viruses include parvoviruses, polyomaviruses, adenoviruses, it is less preferred to use larger DNA viruses such as herpes viruses, since their genome spans 150 kb.

Preferably the viral sequences cloned into the plasmid will be less than 50 kb, typically ranging from 4 kb (e.g. parovins) up to about 36 kb (e.g. adenovirus). RNA viruses suitable for the application of the present invention include, but are not limited to retroviruses such as those in the Retroviridae family (spumaviruses or foamy viruses, lentiviruses and oncoviruses). Lentiviruses include the “immunodeficiency viruses” which include human immunodeficient virus type 1 and type 2 (HIV-1 and HIV-2) and simian immunodeficiency virus (SIV). Oncoviruses are not necessarily oncogenic, and include Mouse mammary tumour virus (MMTV), murine leukaemia viruses (MLV), bovine leukaemia virus (BLV) and human T-cell leukaemia viruses type I and II (HTLV-I/II). Further suitable RNA viruses include picornaviruses, rhinoviruses, cornaviruses, togaviruses, hepatitis viruses and influenza viruses.

It will of course be understood that the invention further extends to modified viruses, such as hybrid viruses that utilise components from varying viruses to produce a virus which has particular desired characteristics. Such hybrid viruses include adenovirus-retrovirus hybrids and adenovirus-retrotransposon hybrid, but any suitable hybrid may be used.

Further, it may be possible to produce a “designer” virus via recombination of various coding sequences and regulatory sequences from a number of viruses.

The virus will typically be recombinant in that its genome is the product of manipulation by recombinant nucleic acid technology and not the same as the wild type genome. The genome will therefore generally incorporate a region of heterologous nucleic acid, i.e. a region which does not normally exist in the wild type virus, including native regions which have been modified to alter their function. Preferably the heterologous region will encode a therapeutic molecule such as a suicide gene (e.g. PNP), or a therapeutic protein (e.g. an immunostimulatory or anti-inflammatory cytokine) or dominant-negative molecules, siRNA, antisense molecules etc.

The plasmid of the invention thus contains sequences which encode a replication-competent virus. The elements which comprise the plasmid will vary dependent upon the identity of the virus encoded thereby. As hereinbefore described, the plasmid comprises the following elements:

the viral nucleic acid sequences required for;

(i) the production, assembly, and release of a virion particle, such as the E1B-19 KD gene isoform of adenovirus which helps to shut off host cell protein synthesis and enhances apoptosis in the later stages of infection, thus promoting viral release; and structural genes such as the env (envelope proteins) gene of retroviruses and the “late” genes of the adenovirus L1, L2, L3 and L4 which encode the structural components of the viral capsid, such as the hexon, penton and fiber units;

(ii) the packaging of said virus sequences (referred to herein as the “viral genome”) within said particle, such as the Psi retroviral packaging sequence;

(iii) the cellular entry and establishment of infection by said particle, such as the E1B-55 KD isoform of adenovirus which encodes a protein which inhibits the cellular defence protein p53, which would otherwise inhibit DNA replication, halting viral infection; and

(iv) the replication of the viral genome sequences within the infected cells, such as the “Early” gene E1A of adenovirus which is the master transcriptional activator for virus replication, or the pol gene of retroviruses which encodes reverse transcriptase, protease and integrase proteins.

It will be understood that the plasmid thus contains at least the minimum sequences required in order to produce within the transfected cell a fully replication-competent recombinant virus. Some of the sequences present in the plasmid may perform more than one function, i.e. fulfil more than one of the roles identified as (i) to (iv). It will further be understood that the sequences performing the above-mentioned roles need not necessarily be “gene or coding” sequences encoding a peptide, but may be promoter or enhancer regions within the nucleic acid sequence. The sequences included in the plasmid include but are not limited to terminal repeat sequences and packaging signal sequences, as well as sequences encoding wild type or heterologous viral transcription factors, polymerases, and structural capsid and/or envelope proteins, with operably linked regulatory sequences such as wild type or heterologous promoters or enhancers.

The retroviral genome is fairly simple and it, or variants of it, are particularly preferred for incorporation into the plasmids of use in the present invention. It comprises only 3 gene loci; gag (encoding capsid and matrix proteins), pol (encoding reverse transcriptase) and env (encoding viral envelope glycoproteins). These structural genes are flanked by two long terminal repeat (LTR) sequences which serve to provide transcription and polyadenylation of the virion RNAs and contain all other cis-acting sequences necessary for viral reproduction, including a packaging signal sequence.

If it is desired that the recombinant virus introduced into the cell via transfection with a plasmid is to deliver a therapeutic gene to a target cell, such a therapeutic gene (or coding sequence) is included in the plasmid, as part of the viral genome. However, in some embodiments, it will not be desired to deliver any therapeutic gene to the target cell. Instead, a lytic virus may be encoded by the plasmid, and once this virus is produced in the cell, it destroys the cell by promoting apoptosis in order to release the progeny viruses, for example adenovirus is a lytic virus. This strategy may be used, for example, to treat cancer cells. Once the plasmid has been transfected into a cancer cell, and the lytic virus expressed, the cancer cells then dies as the progeny viruses are released. Such viruses are known as “cytolytic” viruses, and are within the scope of the present invention. Such viruses may be targeted to cancer cells, as discussed further below.

Alternatively, the plasmid may contain a therapeutic gene or coding sequence which it is desired to introduce into the target cells. This can be a gene encoding any desirable therapeutic agent, such as non-mutated versions of normal host proteins, such as insulin, growth hormones, blood clotting factors, cytokines (such as interleukins), or alternatively, genes encoding suicide genes such as enzymes capable of converting prodrugs, ribozymes, antisense RNA, siRNA (small inhibitory RNA for gene silencing) and modifiers/enhancers/suppressors of gene expression. The person skilled in the art will be aware of suitable genes for inclusion in the plasmid. The therapeutic gene/coding sequence will generally be operably linked to a promoter which will allow transcription of the gene once inside the cell. Optionally, the therapeutic gene is also linked to an enhancer. Both the promoter and enhancer may be those naturally present in the virus, those normally associated with the gene or coding sequence in its natural form, or can be exogenous elements provided from any suitable source. In a preferred embodiment of the invention, the promoter and/or enhancer sequences controlling expression of the therapeutic gene are specific for the cells to which the therapy is targeted (the target cell), thus allowing expression of the therapeutic gene or coding sequence only in the target cell population. Such targeting will be discussed further below.

In the use described herein, the plasmid may contain none, one or more (e.g. 1, 2, 3, 4 or 5) therapeutic genes for delivery to the target cell.

The plasmid utilised in the invention contains the necessary sequences to allow transcription and translation of the viral genes/coding sequences to take place, together with the therapeutic coding sequences, if present. Hereinafter such sequences as described as a “regulatory nucleic acid sequence” and include promoter sequences, polyadenylation signals, transcription termination sequences, upstream regulatory domains, replication origins, internal ribosome entry sites (IRES), enhancers and the like, which collectively provide for the replication, transcription and translation of a coding sequence in a cell. The natural viral sequences may be used. It is not necessary for all of these elements to be present in order for the coding sequences to be replicated, translated and transcribed. However, in order to increase target cell specificity, it is possible to use non-heterologous sequences to direct coding sequence expression from the plasmid. For example, it is possible to use tissue specific promoters and/or enhancers or any other sequence which modifies the level of expression of the genes contained in the plasmid. Thus, mention can be made of tumour associated promoters and enhancers such as MUC-1, PSA and tyrosinase, and tissue specific promoters and/or enhancers such as those for the glucagon gene promoter, which restricts gene expression to gut endocrine cells.

The progeny virus resulting from transfection of a cell with a plasmid may rely upon their natural cell-binding abilities (tropism) to infect the target cells. More preferably, the tropism of the viruses can be enhanced or altered by modifying the gene/coding sequence for the protein responsible for target cell binding and entry. Such re-targeted viruses are well known in the art. For example, the envelope proteins of retroviruses can be modified to alter tropism. Thus, the coding sequence for the coat or envelope protein responsible for cell targeting of any virus may be modified, preferably by addition of a targeting nucleic acid sequence. The targeting nucleic acid sequence codes for a targeting ligand, such as an antibody and derivatives thereof (such as single-chain antibodies), antigens, lectins, glycopeptides, peptide hormones such as heregulin, receptors or ligands for a receptor (such as the binding pair biotin and avidin). However, any targeting moiety could be used.

The initial plasmid transfection step may occur in any cell, it need not be the target cell for viral therapy as discussed further below. The ‘initial cell’ is the cell transfected by the administered plasmid. The initial transfected cell may thus be any cell, but preferably it is a target cell, or in the same organ or tissue as the target cell, such that the viral-therapy is directed to the area of need. Thus, the initial cell may be, for example, an easily accessible place for plasmid transfection i.e. on the surface of an organ such as the liver, wherein the target cell is less accessible, i.e. within the liver core. Preferably however, the initial cell to be transfected is the target cell, or is in close proximity thereto.

The plasmid may preferably be associated with, e.g conjugated to, a targeting moiety or ligand which directs the plasmid to a specific type of tissue or cell and hence promotes transfection of said specific tissue or cell type. For example, a targeting moiety may be used to specifically target tumour cells.

The “target cell” is the cell to which the viral-therapy is directed. A cell may be a target cell purely on the basis of cell type, e.g. liver cell, and/or on the basis of location, e.g. smooth muscle cells in the leg. The target cell may be a cancer cell. Alternatively, the target cell may be a cell harbouring an infection such as hepatitis C, a cell involved in the inflammation process, or a cell that requires the provision of an externally provided gene, such as pancreatic cells in a diabetic patient may be provided with a functioning coding sequence for insulin. Thus, the target cell may be one which it is desired to destroy or in which it is desired to alter a given property, by transferring a coding sequence to that cell which will directly or indirectly reduce, ameliorate or treat a condition associated with that cell.

The plasmid encoding the replication-competent virus may be used to treat any disease, condition, disorder, infection or inflammation. Particularly, the present invention can be used to treat any cell proliferative disease, such as cancers, immunological diseases such as SCID, neuronal disorders, such as Parkinson's, acquired infections, such as Hepatitis C infection and inflammation. Those skilled in the art will be aware of the scope of conditions and diseases that may be treated using gene, particularly viral based therapy.

Further, the present invention could be used for immunization purposes, for example, the plasmid encodes a replication-competent virus against which it is desired to raise antibodies.

In addition to the replication-competent virus sequence, the plasmid of the invention may comprise a plasmid backbone. Suitable plasmid backbones will be known to the person skilled in the art and are described in common textbooks such as Sambrook et al. (Molecular Cloning, A laboratory Manual, second edition, Cold Spring Harbor laboratory Press). The plasmid backbone preferably possesses an origin of replication to allow replication of the plasmid in a cell culture. In this context, “cell culture” means an in vitro cell culture in which a plasmid may be propagated and maintained and does not refer to the cells of the subject to which a plasmid of the invention is administered for therapeutic purposes. Typically, the cell culture will be a bacterial cell culture, e.g. a suitable strain of E. coli, but other cell cultures including yeast may be used. The plasmid origin of replication must be compatible with the cells of the cell culture, and the skilled person will be aware of how to chose appropriate combinations.

Examples of suitable plasmid backbones include ColE1-type plasmids such as pBR322 (available e.g. from TopoGEN, Inc. 108 Aces Alley Port Orange, FL, USA 32128) which contain the ColE1 origin of replication, pUC18 (GenBank/EMBL accession number L09136), pUC19 (GenBank/EMBL accession number L09137), R1 plasmids containing oriR (Nordstrom K, Molin S, Light J. Control of replication of bacterial plasmids: genetics, molecular biology, and physiology of the plasmid R1 system. Plasmid. 1984 September; 12(2):71-90.) and plasmids containing the pMB1 and/or p15A origin of replication.

Another example of a suitable plasmid backbone is R6K, available e.g. from DSMZ-Deutsche Sammlung von Mikroorganismen und Zellkulturen GmbH, Braunschweig, Germany. R6K possesses three origins of replication, alpha, beta and gamma and the pir gene which encodes pi protein required for replication of R6K.

A plasmid according to the present invention may be constructed by combining sequence from a suitable replication-competent virus with sequence from a suitable plasmid. Alternatively, the desired plasmid and/or virus sequence may be synthesised de novo.

Preferably, the origin of replication is one which yields a high copy number of plasmid per host cell, e.g. ColE1, to allow recovery of high amounts of plasmids per host cell, but in some instances a low copy number origin of replication may be preferred.

The plasmid backbone preferably also contains one or more selectable markers to allow identification/selection of those cells which have been transformed with the plasmid. Suitable selectable markers are known to the skilled person. Preferably, the marker is an antibiotic resistance gene which confers resistance to e.g. ampicillin, kanamycin, tetracycline, bleomycin or the like. Other examples of suitable selectable markers include heavy metal resistance genes and amino acid biosynthesis markers.

The plasmid may be constructed using routine recombinant nucleic acid technology, such as cutting of desired nucleic acid fragments with restriction endonuclease, and ligation of nucleic acid fragments using, for example, DNA ligase. Where the original viral sequence is RNA, and it is wished to use a DNA plasmid, it is possible to use reverse transcriptase in order to generate the DNA sequence corresponding to the RNA sequence for use in the vector. In the case of some RNA viruses, it is possible to use the cDNA sequence in the construction of the plasmid. Plasmid construction methods are well known in the art and are described in Sambrook, Fritsch and Maniatis, Molecular Cloning, Cold Spring Harbour Laboratory Press.

The plasmid is used directly in vivo in the invention. Thus, the plasmid encoding the recombinant replication-competent virus is itself introduced into the target organism. The statement above that the plasmid is “for use in therapy” must be interpreted with this in mind. Any method or use where a plasmid as described herein is generated in a therapeutic context but is not administered to a patient, for example because the virus encoded by the plasmid is administered instead, is not within the scope of the present invention. Such delivery of plasmids encoding viruses has not previously been contemplated, and overcomes the cumbersome methods of the prior art in cultivating sufficient titre of viruses for initial transfection events.

Alternatively viewed therefore the invention provides a plasmid encoding a replication-competent virus for use in the in vivo production of replicative viruses.

Further, the invention provides a plasmid encoding a replication-competent virus for the delivery of therapeutic genes to target cells/tissue/organ.

As previously mentioned, the present invention does not require the cumbersome purification of virus particles; instead, a plasmid is used to transform cells of the subject in need of therapy. In one embodiment, the plasmid may be produced by transforming a suitable cell, typically a baterial cell, with the plasmid, culturing the cell under conditions which favour maintenance and/or replication of the plasmid and purifying the plasmid from the cell culture using standard methods. The plasmid may then be used directly to transform cells of the subject in need of therapy. This embodiment is preferred for RNA viruses.

In another embodiment of the invention, preferred for DNA viruses, the virus sequence is initially inserted into a suitable vector such as a plasmid, cosmid, bacterial artificial chromosome (BAC), or yeast artificial chromosome (YAC). The desired quantity of such a vector plus virus sequence construct may then be produced by transforming a suitable host cell (typically a bacterial cell, but yeast in the case of a YAC) and culturing the host cell. The vector plus virus sequence construct may then be purified from the cells and the virus sequence may be separated from part or all of the vector, e.g. via restriction enzyme digestion or by using a recombinase. The desired nucleic acid molecule comprising the virus sequence may then be purified by standard methods, e.g. gel electrophoresis or chromatography. The nucleic acid molecule comprising the virus sequence obtained in this way may then be used as a plasmid of the present invention. This plasmid may be linear or circular. In some instances, a linear plasmid may be preferred, e.g. when the virus naturally occurs as a linear nucleic acid molecule, e.g. adenovirus. Preferably, the ends of the linear nucleic acid molecule may be protected, e.g. by conjugating them with a recombinant form of the viral terminal binding protein TBP prior to transfection into the subject.

In other instances, a circular plasmid may be preferred, and any linear nucleic acid molecule may be treated to circularize it. In this embodiment, the initial construct preferably contains specific and unique restriction endonuclease or recombinase recognition sequences (e.g., rare cutting restriction enzymes such as Pme I, or loxP sites for CRE recombinase) at the terminal ends of the linear sequence of the inserted virus.

Thus in a further aspect, the present invention provides a method of producing a plasmid encoding a replication-competent virus for use in therapy, said method comprising:

(a) providing a vector comprising nucleic acid encoding a replication-competent virus in a host cell;

(b) culturing said host cell; and

(c) recovering said plasmid.

In one embodiment, the method further comprises

(d) excising a nucleic acid fragment encoding said replication-competent virus;

(e) purifying said fragment

Preferably, the host cells used in the above methods are not packaging cells (cells which contain integrated wild type virus genome sequences and thus provide all of the structural elements necessary to assemble viruses, but cannot encapsidate their own wild type virus genomes due to a deletion of the packaging signal sequence psi).

The target organism may be a non-human animal, such as a mammal, bird, reptile or fish. Preferably, the plasmid is used directly in vivo in mammals, such as companion animals (dogs and cats), livestock animals (such as cattle, horses, sheep and goats), or non-human primates such as monkeys and gorillas. Most preferably however, the target organism is a human.

The plasmid is transfected into cells (which as discussed above, may or may not themselves be target cells) in vivo using any suitable methodology. Thus, the plasmid may be transfected into the initial cell via, e.g. lipofection, electroporation or ballistic gene transfer methods. Further, chemical agents which induce transfection may also be introduced in vivo with the plasmid. Typically the transfection agent will be included in a combined preparation containing the plasmid or it may be administered simultaneously or sequentially.

Thus in a further aspect the invention provides a formulation comprising a plasmid encoding a replication-competent virus together with a transfection agent. As discussed in more detail below, as well as chemicals the transfection agent may be in the form of pellets (e.g. tungsten micropellets) when ballistic gene transfer is used. Transfection agents include carriers suitable for use in transfection. Examples of suitable transfection agents include formulations of lipid compounds that can be mixed with DNA to facilitate its uptake by mammalian cells, e.g. Lipofectin, Lipofectamine, Fugene, DOTAP, DMRIE, DC-Chol. These are known to the skilled person, and many are commercially available. Polymers such as polyethylenimine (PEI), or peptide ligands containing polycationic sequences for electrostatic conjugation with DNA to form “polyplexes” may also be used.

In the case of transfection via electroporation methods (in which the tissue or organ is bathed in an electrolyte/plasmid solution and about 2000 Volts of electricity are applied, opening large holes in the cell and nuclear membrane, which the plasmid can then pass through), it will of course be more desirable for the cells involved in the initial transfection event to be easily accessible.

Particle bombardment methods (also known as “ballistic gene transfer”) may also be used to transfect the initial cell with the plasmid, in which a so-called “gene gun” is used to shoot plasmid-conjugated tungsten micropellets into cells and tissues at high velocity. Such techniques can readily be used in vivo.

Alternatively, the plasmid can be introduced in vivo with physiologically acceptable chemical transfection agents such as lipofecting agents, which may be cationic or amine based. Lipofection involves the plasmid and liposome complex undergoing endocytosis into the cell. Since most of the complex that enters the cell will be degraded in lysosomes, it is generally necessary to transfect multiple (i.e. one thousand, 10 thousand, 100 thousand) copies of the plasmid such that some escape lysosome degradation and enter the nucleus by bulk flow even in the absence of any mechanism for active transport. Other chemicals that may be used include calcium phosphate.

In addition, there is a procedure known as “hydrodynamic transfection” in which a large fluid volume of plasmid solution is delivered into the vasculature at high pressure, and the probable combination of barotraumas and longer contact time of the plasmid with the initial cells (because the large fluid volume takes longer to drain away) results in relatively good levels of transfection. A suitable method of hydrodynamic gene delivery in mice has been reported by Zhang et al. 1997. This involves infusion of the plasmid solution through a 27-gauge needle placed in the tail vein. In larger animals and humans, injections for hydrodynamic plasmid delivery to the liver may also be performed via (a) portal vein injections, in which case outflow is transiently blocked during and immediately after the infusion procedure by occluding the hepatic vein and inferior vena cava, or (b) via hepatic vein injections (via the inferior vena cava), in which case outflow is transiently blocked during and immediately after the infusion procedure by occluding the portal vein, vena cava, and hepatic artery, or (c) hepatic artery injections (via the femoral artery and abdominal aorta), in which case outflow is transiently blocked during and immediately after the infusion procedure by occluding the hepatic vein and portal vein. Although hydrodynamic transfection is described above with reference to the liver, the skilled person will appreciate that equivalent methods can be used to transfect other organs or parts of the body.

Suitable physiologically acceptable carriers or diluents which may also be included in the formulations are known to the skilled man.

It is possible to enhance the rate of transfection and entry into the nucleus by direct targeting of the plasmid. Plasmid-protein conjugates may generally be used, for example polylysine can be used in plasmid targeting strategies. A number of groups have reported enhanced efficiency of plasmid transfection and expression when proteins containing nuclear localization sequences are pre-bound to the plasmid DNA (for example, high mobility group (HMG) non-histone proteins have been used, and transcription factors have been used to enhance plasmid entry into the nucleus).

The present invention thus relates to the direct use in vivo of a plasmid to deliver to a cell the coding sequences of a recombinant replication-competent virus, optionally incorporating a therapeutic gene/coding sequence.

The present invention thus extends to a method of treatment of a human or animal patient comprising the in vivo transfection of a cell of said patient with a plasmid coding for a replication-competent virus. Typically the viral genome will incorporate a therapeutic gene for the treatment of a condition which the patient is suffering from but alternatively the virus may itself ‘treat’ the patient e.g. wherein the virus causes lysis of transfected cells and thus destruction of a solid tumour. Preferably the patient is human. ‘Treatment’ includes partial or total amelioration of the patient's condition or of one or more symptoms thereof.

Once the plasmid has been transfected into a cell, and the plasmid has entered the nucleus, the cellular transcription and translation machinery should commence transcription and translation of the coding sequences contained within the plasmid. Once the plasmid has entered the nucleus, that cell is effectively infected by the virus which is coded for by the plasmid, and the viral life cycle commences when the plasmid coding sequences are translated and transcribed by the host cellular machinery.

Since the viruses encoded by the plasmid are replication-competent, at a suitable point in the life-cycle, recombinant replication-competent viruses will be released from the initially transfected cell. Whether the initially transfected cell is lysed during the release of the progeny viruses depends on the nature of the virus. If the virus is lytic, such as adenovirus then release of progeny virus will be concomitant with cell lysis. However, some viruses bud harmlessly from the cell surface, such as MLV, and thus the initially transfected cell will survive.

Thus, the initially transfected cell acts essentially as an in vivo virus producing cell, and the viruses are then released to infect their target cells, and in turn these target cells become viral producing. Thus, from an initial transfection event using a plasmid, multiple transfection events may be achieved.

It may be desirable to utilise a control mechanism in order to halt the spread of the viral vector. Passive or active immunization as a follow up to plasmid-mediated viral therapy may be used, and this would involve either supplying antibodies or viral vaccine to the patient involved. Such therapy should terminate viral spread and provides a further safeguard which will minimise any risks to non-target cells. Anti-viral drugs, such as the anti-retroviral drug AZT (azido-3′-deoxythymidine) can readily terminate viral replication and spread. Further “suicide” genes, such as those already mentioned with regard to therapy, may be inserted into the viral genome should it be desired. Alternatively the regulatory nucleic acid sequences for key structural viral components may be made dependent upon exogenously supplied materials, such as tetracycline, by the use of a tetracycline inducible promoter. Other such inducible promoters include rapamycin/FK 506-binding protein inducible promoters. Thus, once the exogenously-supplied inducer is withdrawn, viral spread is hindered.

In a particularly preferred embodiment of the present invention, the plasmid encodes a (recombinant) replication-competent retrovirus. The incorporation of nucleic acid sequences for targeting certain cell types is preferable, since this reduces or eliminates native pathogenicity whilst improving target specificity. A particularly preferred retroviral construct has been discussed in U.S. Pat. No. 6,410,313 which is incorporated herein by reference.

In one preferred embodiment, the present invention thus provides a plasmid encoding a replication-competent retrovirus comprising a retroviral GAG coding sequence; a retroviral POL coding sequence; a retroviral ENV coding sequence; retroviral Long Terminal Repeat (LTR) sequences; and optionally one or more of the following elements; a heterologous coding sequence operably linked to a regulatory nucleic acid sequence; one or more targeting sequences for cell- or tissue-specific targeting of the retrovirus.

The target specific nucleic acid sequence as discussed previously may be a tissue or cell-type specific promoter or enhancer sequence, such as heregulin promoter sequences. This is generally placed at the 5′ and or 3′ end of the viral genome. To target the retrovirus to a specific target cell or tissue, the retroviral ENV coding sequence may be modified to further comprise a target—specific ligand or binding moiety as hereinbefore discussed.

Since the sequences required for encapsidation are provided in the plasmid the virus formed is replication-competent.

The plasmid thus has at least three genes; the gag, the pol, and the env genes, which are flanked by two long terminal repeat (LTR) sequences containing cis-acting sequences such as Psi, which is responsible for efficient encapsidation of viral RNA, and sequences necessary for reverse transcription of the genome, such as the tRNA primer binding site.

The gag gene encodes the internal structure (matrix, capsid and nucleocapsid) proteins; the pol gene encodes the RNA-directed DNA polymerase (reverse transcriptase), protease and integrase; and the env gene encodes viral envelope glycoproteins. The 5′ and 3′ LTRs serve to promote transcription and polyadenylation of the virion RNAs. The LTR also contains other cis-acting sequences necessary for viral replication. Lentiviruses have additional genes including vif, vpr, tat, rev, vpu, nef and vpx (in HIV-1, HIV-2 and/or SIV).

The tissue or cell specific regulatory element (i.e. enhancer/promoter), if present, is preferably linked to the 5′ and/or 3′ LTR, creating a chimeric LTR.

In the plasmid described herein, the heterologous (typically therapeutic) coding sequence is preferably under the control of either the viral LTR promoter-enhancer signals or an internal promoter. Accordingly, the desired sequence can be inserted at several sites and under different regulatory regions. For example, a site for insertion can be the viral enhancer/promoter site (i.e. the 5′ LTR—driven gene locus). Alternatively, the desired sequence can be inserted into a regulatory distal site e.g. the IRES (internal ribosome entry sites) sequence 3′ to the env gene).

Thus, in one embodiment, the retroviral plasmid used according to the present invention contains an IRES comprising an insertion site for a desired nucleic acid sequence such as a heterologous sequence, preferably the IRES is 3′ to the env gene in the retroviral vector. Accordingly, a heterologous nucleic acid sequence, for example, encoding a heterologous polypeptide may be operably linked to the IRES. An example of nucleic acid sequence which may be operably linked to the IRES are suicide genes, such as PNP and HSV-thymidine kinase, sequences that encode an antisense molecule, or sequences that encode a ribozyme.

The viral gag, pol and env genes or coding sequences can be derived from any suitable retrovirus (e.g. MLV or lentivirus-derived, i.e. HIV or MOMLV). In an alternative embodiment, the viral ENV gene is non-retrovirus-derived (e.g., CMV or VSV). The env gene can be derived from any retroviruses. The env may be an amphotropic envelope protein which allows transduction of cells of human and other species, or may be an ecotropic envelope protein, which is able to transduce only mouse and rat cells.

In a preferred embodiment, the plasmid of the invention contains the full sequence of the replication-competent amphotropic murine leukemia virus (MLV) vector construct. More preferably, it contains the full sequence of the replication-competent amphotropic murine leukemia virus (MLV) vector construct, in which the cytomegalovirus (CMV) promoter has been used to replace the 5′ long terminal repeat (LTR) U3 region, and an encephalomyocarditis virus internal ribosome entry site (IRES)—therapeutic gene expression cassette is inserted between the env gene and 3′ LTR.

Further, it may be desirable to target the recombinant virus by linkage of the envelope protein with an antibody or a particular ligand for targeting to a receptor of a particular cell-type. As mentioned above, retroviral vectors can be made target specific by inserting, for example, a glycolipid, or a protein. Targeting is often accomplished by using an antibody to target the retroviral vector to an antigen on a particular cell-type (e.g., a cell type found in a certain tissue, or a cancer cell type). Those of skill in the art will know of, or can readily ascertain without undue experimentation, specific methods to achieve delivery of a retroviral vector to a specific target. In one embodiment, the env gene is derived from a non-retrovirus (e.g., CMV or VSV). Examples of retroviral-derived env genes include, but are not limited to: Moloney murine leukemia virus (MoMuLV), Harvey murine sarcoma virus (HaMuSV), murine mammary tumour virus (MuMTV), gibbon ape leukemia virus (GaLV), human immunodeficiency virus (HIV) and Rous Sarcoma Virus (RSV). Other env genes such as Vesicular stomatitis virus (VSV) (Protein G), cytomegalovirus envelope (CMV), or influenza virus hemagglutinin (HA) can also be used. Thus, the skilled man can construct a hybrid vector utilizing different genes from different viruses in the design of the plasmid for use in the invention. Similar targeting methods are suitable for different viruses.

Cell or tissue specific regulatory nucleic acid sequences (e.g., promoters) can be utilized to target expression of gene sequences in specific target cell populations. Suitable mammalian and viral promoters for the present invention are known to those skilled in the art. Accordingly, in a preferred embodiment, the present invention provides a plasmid having a tissue-specific promoter element at the 5′ and/or 3′ end of the viral genome. Preferably, the tissue-specific regulatory elements/sequences are in the U3 region of the LTR of the retroviral genome, including for example cell—or tissue—specific promoters and enhancers to cancerous cells (e.g., tumour cell-specific enhancers and promoters), and inducible promoters (e.g., tetracycline). Transcription control sequences of the present invention can also include naturally occurring transcription control sequences naturally associated with the heterologous gene.

Once the plasmid has been transfected into the initial cell, and the progeny viruses have been released, the recombinant replication-competent retroviruses are able to infect further cells, preferably their target cells. After infection of a cell by the virus, the virus injects its nucleic acid into the cell and the genetic material can integrate into the target cell genome. The transferred genetic material is then transcribed and translated into proteins within the host cell. The inserted heterologous (e.g. therapeutic) coding sequence in the plasmid will be transferred to the target cell nucleus and may integrate into the target cell DNA.

Some classes of retroviruses have the ability only to infect dividing cells, since they lack the necessary signals to transfer their genetic material across the nuclear membrane at any time, and thus must wait for the nuclear membrane to dissolve during mitosis. Class C-type retroviruses, such as spleen necrosis virus (SNV) are examples of such viruses. Thus, these are preferred viruses to use in delivering genes to target cells with a cell proliferation disorder. Such disorders include any condition characterised by abnormal numbers of cells and active cell division. Thus, such conditions include all types of cancer, but the cell populations are not necessarily transformed, tumorigenic or malignant, but can include normal cells as well. Cell proliferation may occur during inflammation and infection, or during conditions such as cirrhosis of the liver. Some cell populations, such as skin cells, are continuously regenerating and thus may be targeted using retroviruses that may only transfect dividing cells.

In the case of unwanted proliferation events, such as cancer, the virus may deliver a suicide gene, such as the Herpes Simplex thymidine kinase (HSV-tk) gene and the E. Coli purine nucleotide phosphorylase (PNP) genes. Alternatively, the virus may deliver a regulator of the cell-cycle, an anti-inflammatory cytokine, such as an interleukin, a ribozyme to recognise a particular malignancy-related RNA and cleave it or anti-angiogenesis factors. Many suitable therapeutic coding sequences are known to those skilled in the art. It will be appreciated that such heterologous coding sequences may also be carried by any of the viruses mentioned herein.

Retroviridae have an RNA genome which acts as a template for the production of viral DNA. This is achieved by RNA dependent DNA polymerase (reverse transcriptase) that is packaged with the RNA genome. The resulting viral DNA integrates into the host cell genome to provide the template for viral RNA synthesis by host derived mechanisms. Thus, to produce a DNA plasmid coding for a retrovirus, it is possible to use reverse transcriptase to produce a viral DNA copy of the viral RNA sequence. Such DNA sequences may be modified if desired.

In a particularly preferred embodiment, the plasmid encodes a recombinant replication-competent murine leukaemia virus (MLV), comprising a MLV gag coding sequence; a MLV env coding sequence; a MLV pol coding sequence; a MLV nucleic acid sequence comprising LTR sequences at the 5′ and 3′ end of the retroviral genome; cis acting nucleic acid sequences necessary for reverse transcription, packaging and integration in a target cell, and optionally a heterologous coding sequence operably linked to a regulatory nucleic acid sequence.

Gene therapy vectors based upon MLV have been described in the art (Logg et al, Journal of Virology, December 2002, 12738 to 12791; Logg et al, Journal of Virology, August 2001, 6989 to 6998 and Tai et al, Human Gene Therapy 14:789 to 802, May 2003).

1. The following paragraphs enumerated consecutively from 1 through 16 provide for various aspects of the present invention. In one embodiment, in a first paragraph (1), the present invention pertains to a plasmid encoding a replication-competent virus for use in therapy.

2. A plasmid according to paragraph 1 for use in the treatment of a cell proliferative disease, an immunological disease, a neuronal disorder, an acquired infection and/or inflammation.

3. A plasmid according to paragraph 2, wherein the disease is selected from cancers, SCID, Parkinson's, Hepatitis C infection and/or Diabetes.

4. A plasmid according to any one of paragraphs 1 to 3, wherein the replication-competent virus is a retrovirus.

5. A plasmid according to paragraph 4, wherein the replication-competent retrovirus comprises:

a retroviral GAG coding sequence;
a retroviral POL coding sequence;
a retroviral ENV coding sequence;
retroviral Long Terminal Repeat (LTR) sequences and optionally one or more of the following elements;
a heterologous coding sequence operably linked to a regulatory nucleic acid sequence;
one or more targeting sequences for cell- or tissue-specific targeting of the retrovirus.
   6. A plasmid according to any one of paragraphs 1 to 5, wherein the plasmid contains a therapeutic gene and/or coding sequence.
   7. A plasmid according to paragraph 6 wherein the therapeutic gene and/or coding sequence is operably linked to a promoter and/or enhancer specific for the cells to which the therapy is targeted.
   8. A plasmid according to any one of paragraphs 1 to 5, wherein the replication competent virus is a lytic virus.
   9. A plasmid according to paragraph 8, wherein the lytic virus is adenovirus.
   10. A plasmid according to any one of paragraphs 1 to 9 wherein the tropism of the virus is enhanced or altered.
   11. A plasmid according to any one of paragraphs 1 to 10 wherein the method used to deliver the plasmid to a subject in need of therapy is hydrodynamic transfection.
   12. A formulation comprising a plasmid according to any one of paragraphs 1 to 9 together with a transfection agent.
   13. A method of producing a plasmid encoding a replication-competent virus for use in therapy, said method comprising:

(a) providing a vector comprising nucleic acid encoding a replication-competent virus in a host cell;

(b) culturing said host cell; and

(c) recovering said plasmid.

14. A method of treatment of a human or animal patient comprising the in vivo transfection of a cell of said patient with a plasmid coding for a replication-competent virus.

15. A method as in paragraph 14 wherein the viral genome incorporates a therapeutic gene or coding sequence suitable for the treatment of a condition which the patient is suffering from.

16. A method as in paragraph 15 wherein said condition is selected from the group comprising a cell proliferative disease, an immunological disease, a neuronal disorder, an acquired infection and inflammation.

The invention will now be further described in the following non-limiting Examples and with references to the Figures in which:

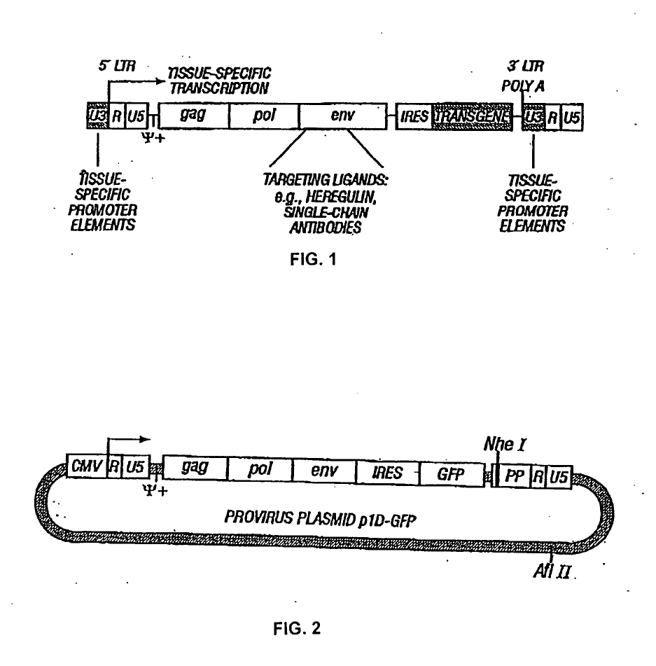

FIG. 1: is a schematic diagram of the structure of a nucleic acid molecule encoding a replication-competent MOMLV retrovirus;

FIG. 2: is a plasmid encoding a replication-competent retrovirus. The target cell for the replication-competent retroviruses are prostate cancer cells;

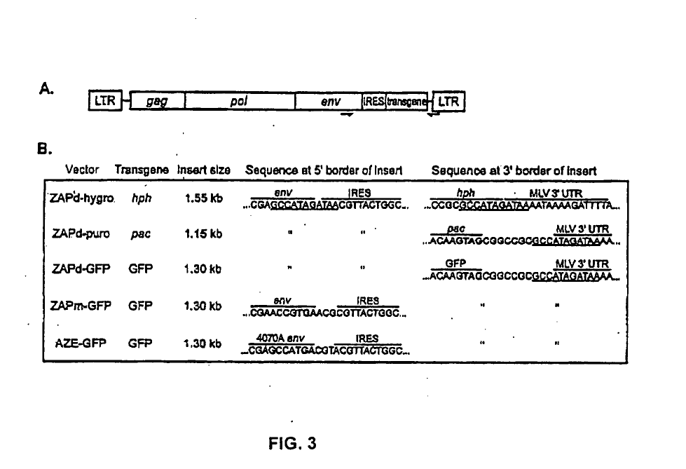

FIG. 3: FIG. 3A shows the general structure of nucleic acid encoding a replication-competent retrovirus, and FIG. 3B shows particular plasmid vectors, indicating the identity of the transgene insert and the sequence at both ends of the transgene insert. Nucleotides shown in bold show the position of the env stop codon.

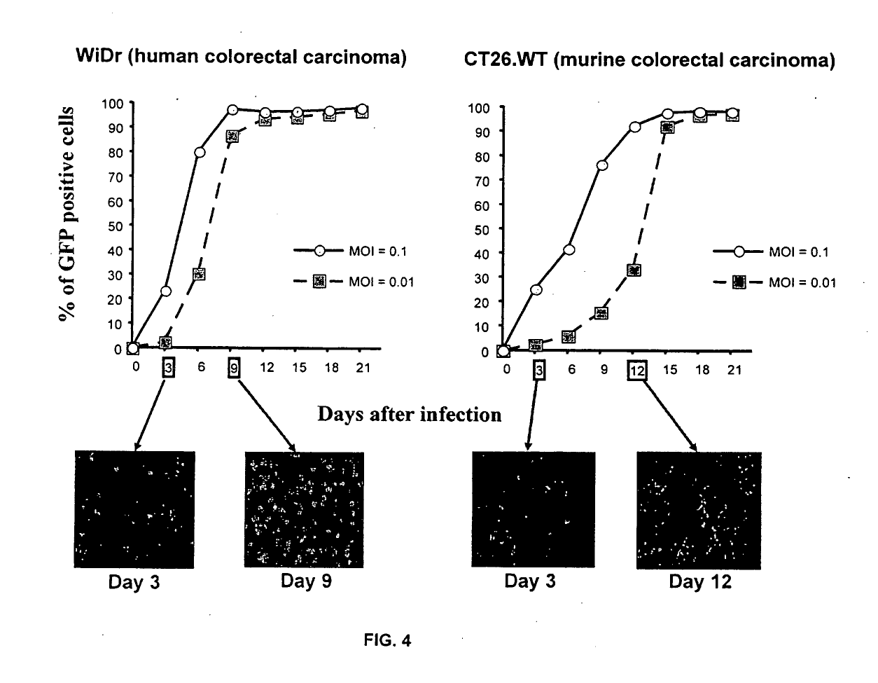

FIG. 4: shows the replication curves of plasmid pACE-GFP-derived retrovirus vectors in the WiDr human colorectal cancer cell line and the CT26.WT murine colorectal cancer cell line, respectively, after inoculation at different doses. The curves in the upper panels show the percentage of GFP-positive cells as determined by flow cytometric analysis every three days following initial inoculation at multiplicities of infection (MOI) of 0.1 and 0.01 (i.e., one infectious nanovector per 10 cells or per 100 cells, respectively). The lower panels show representative images of GFP expression in the infected cells taken by fluorescence microscopy on the indicated days post-inoculation.

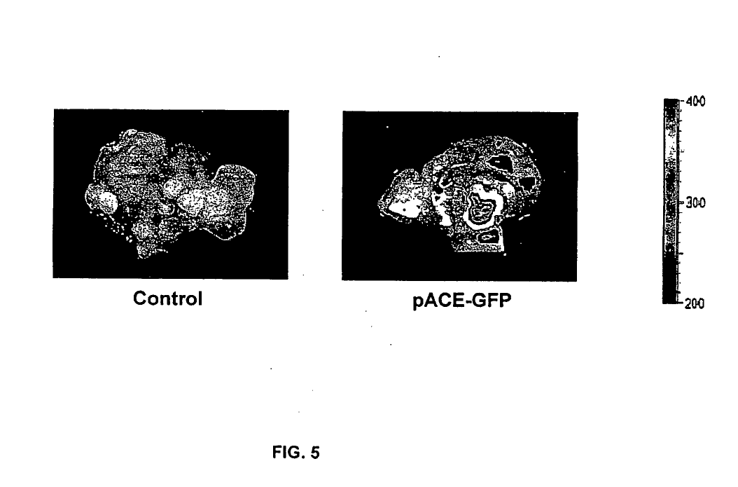

FIG. 5: shows representative composite images after optical imaging of GFP fluorescence from livers isolated 48 hours after hydrodynamic injection of plasmid pACE-GFP (Example 6). The control is shown on the left, GFP expression is shown on the right.

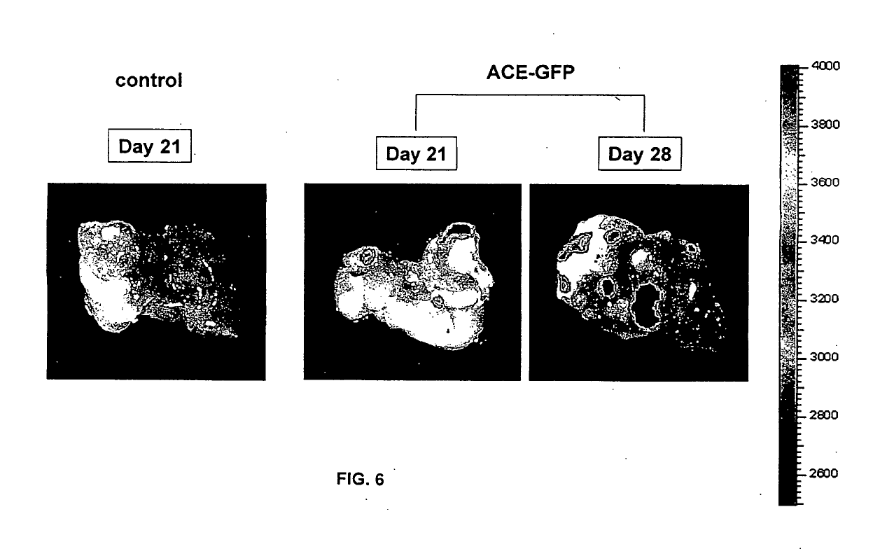

FIG. 6: shows representative composite images after optical imaging of GFP fluorescence from livers isolated on Day 21 (middle panel) and Day 28 (right panel) as described in Example 6. Left panel shows the negative control.

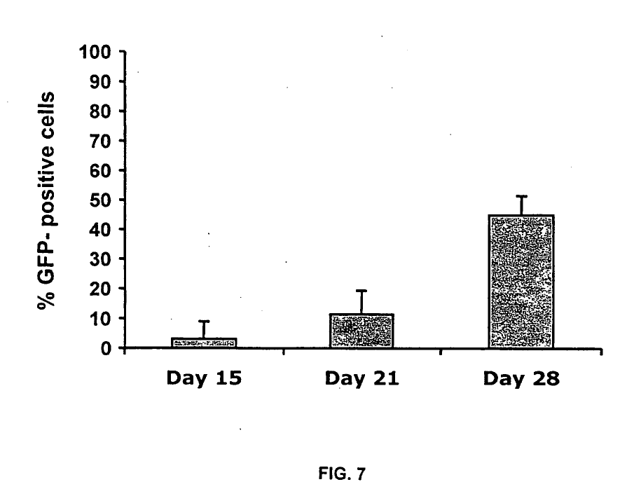

FIG. 7: shows the results of fluorescence-activated cell sorter (FACS) analysis of dispersed tumor cells harvested immediately after dissection at serial time points during pACE-GFP-derived virus nanovector replication in vivo. The largest hepatic tumor of each mouse was removed, digested with collagenase/dispase, and analyzed immediately by FACS (n=4 at each time point). The graph shows the percentage of GFP-positive cells (Y axis) detected in the tumor samples at each time point (X axis).

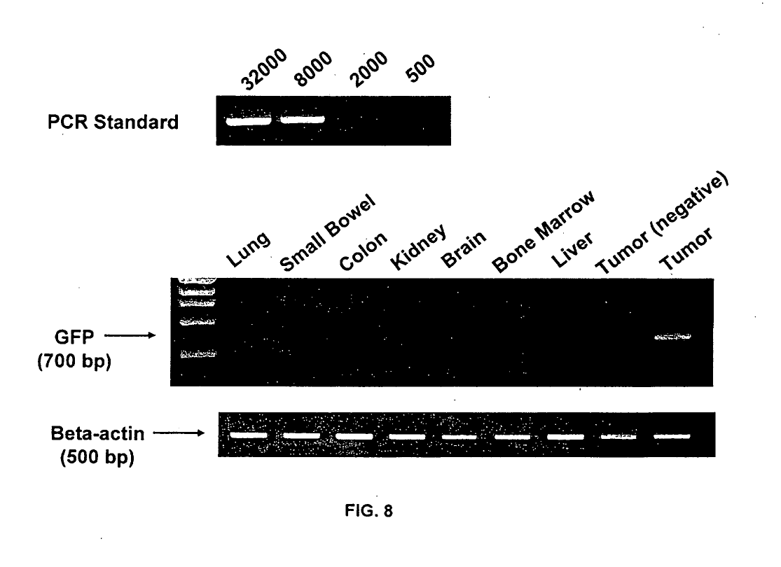

FIG. 8: shows the results from PCR analysis of pACE-GFP-derived replicating virus integration of the GFP transgene.

### EXAMPLES

### Example 1

### Construction of a Plasmid Encoding a Retroviral Virus

An infectious Moloney MLV proviral clone was excised with NheI, which cuts once within each long terminal repeat (LTR), from plasmid pZAP (Soneoka et al, Nucleic Acid Research, 23, 628 to 633) (provided by John A. Young, University of Wisconsin) in order to eliminate flanking rat genomic sequences and recloned in the plasmid backbone of MLV vector g1ZIN to produce plasmid pZAP2. The region of the env gene from the unique NsiI site to the termination codon was amplified by PCR and fused to the encephalomyocarditis virus IRES (Jang et al, J. Virol, 62; 2636 to 2643, 1988) amplified from plasmid pEMCF by overlap extension PCR (Horton et al, Gene; 77, 6028 to 6036), introducing the restriction sites BstBI and NotI at the 3′ end. Plasmids g1ZIN and pEMCF are available from W. French Anderson, University of Southern California.

The region from the env termination codon to the 3′ end of the 3′ LTR was also amplified by PCR, introducing NotI and AflIII sites at the 5′ and 3′ ends of the amplification product, respectively. A three-way ligation was used to insert this PCR product and the overlap extension PCR product into pZAP2 at its NsiI site and an AflIII site in the plasmid backbone, producing plasmid pZAPd. The puromycin acetyltransferase gene (pac) from plasmid PPUR (Clontech), the hygromycin phosphotransferase gene (hph) from plasmid pTK-hygro (Clontech), and the green fluorescent protein (GFP) cDNA (Cormack et al, Gene 173, 33 to 38, 1996) of plasmid pEGFP (Clontech) were each amplified by PCR and inserted into the BstBI and NotI sites of pZAPd, in frame with the authentic start codon of the IRES, producing pZAPdpuro, pZAPd-hygro, and pZAPd-GFP, respectively. All regions generated by PCR were verified by sequencing. A pZAPd-GFP-based construct in which an 11-bp repeat sequence flanking the IRES-GFP insert was eliminated and replaced by an MluI site was also generated by site-directed mutagenesis and designated pZAPm-GFP. An additional construct in which the Moloney MLV ecotropic envelope was replaced with the amphotropic envelope from 4070A was generated by overlap extension PCR and designated pAZE-GFP.

### Example 2

### Good Manufacturing Process (GMP) Grade Plasmid Production

This generally entails the following steps for DNA plasmids for clinical use:
Submission of a Biologics Master File for GMP plasmid production to be reviewed and approved by the relevant regulatory agency (e.g., FDA).
Development of comprehensive Standard Operating Protocols (SOPs) for all GMP operations that adhere to CFR21 (Code of Federal Regulations) and ICH (International Committee on Harmonization) Tripartite Guidelines for Good Manufacturing Practice for Active Pharmaceutical Ingredients
Implementation of SOPs, including master plan validation and validation procedures for all critical equipment, stringent logging and tracking procedures and full quality testing on all incoming raw materials (U.S.Pharmacopoiea (USP) or equivalent grade ingredients and reagents), development of audit schedule for all suppliers, comprehensive training program for all staff involved in GMP procedures, etc.
Per SOPs, production of Master Cell Bank and Manufacturers Working Cell Bank (this consists of generating a clonal E. coli bacterial stock that has been transformed with the plasmid of interest and has been confirmed to replicate the plasmid and maintain it stably; the Master Cell Bank is the initial stock, which is subsequently frozen and stored, while the Working Cell Bank consists of aliquots from the Master Bank that are used for actual production).
Process optimization and development of custom purification procedures specifically designed for scale-up and regulatory compliance prior to GMP production.
The scale-up production process involves expanding the Working Cell Stock (which already has been validated to produce the plasmid) in progressively larger culture scales, until the desired synthesis scale is reached.
For plasmid purification, the bacteria are then pelleted by centrifugation, the supernatant culture medium is removed, chemical/detergent lysis is used to disrupt the bacterial cell wall, and the plasmid fraction is isolated and purified, usually by solvent fractionation and differential centrifugation, or more commonly by resin adsorption and elution or column chromatography.
The final product should be subjected to QC testing and meet at least the following criteria, which will be documented with a Certificate of Analysis for each Lot:
   Low levels of residual endotoxin (<1 EU/mg plasmid)
   Low levels of host cell chromosomal DNA (<1%)
   Low levels of RNA
   Low residual protein
   Low residual solvents
   Predominantly covalently closed circular (supercoiled)
   plasmid (>95%) (In this particular experiment)

### Example 3

### In Vivo Transfection of Plasmid Via Electroporation

Tumor Electroporation Using an Electrode Array:

B16 cells are subcutaneously injected into the flanks of mice (females, 5-6 weeks of age). Unless otherwise stated, 106 cells are injected, and 4 days later, tumors of an average volume of 75 mm³ developed. Plasmid DNA (5.5 μmol) is diluted in 50 micro-litres of K-PBS (30 mM NaCl, 120 mM KCl, 3 mM Na2HPO4, 1.5 mM KH2PO4, 5 mM MgCl2) and injected percutaneously into the tumors by using a syringe with a 27-gauge needle. Tumors are pulsed with an electroporater, CUY21 (Tokiwa Science, Tokyo, Japan) equipped with a 0.5 cm diameter array of seven needle electrodes. In the needle array electrodes, a single center needle is encircled by six needles. Electric current is passed from the center needle to the surrounding needles, or in the opposite direction. Six square-wave pulses are delivered at a frequency of 1s-l, with a pulse length of 100 ms and a voltage of 50 V. Three pulses are followed by other three pulses of the opposite polarity.

Testicular Electroporation for Generation of Gene-Modified Sperm in Transgenic Mouse Production:

ICR strain and [C57BL/6□DBA/2] F1 mice were purchased from SLC, Japan. Postnatal day 14 ICR strain mice were anesthetized with Nembutal solution, and testes were exposed under a dissecting microscope. A micropipet was inserted into the rete testis for injection into seminiferous tubules. Approximately 6-10 μl of the DNA/HBS solution (100-120 μg/ml) was injected into each testis. Electric pulses were delivered with an electric pulse generator (Electrosquare Porator T820, BTX, USA). Testes were held between a pair of tweezers-type electrodes, and square electric pulses were applied four times and again four times in the reverse direction. Each pulse was at 30-50 V and 50 ms in duration.

### Example 4

### Transfection of Plasmid Using Ballistic Gene Transfer

Cutaneous Gene Transfer for DNA Vaccination:

Gene gun particle-mediated DNA vaccination was performed using a helium-driven gene gun (Bio-Rad, Hercules, Calif.) according to the protocol provided by the manufacturer. Briefly, DNA-coated gold particles were prepared by combining 25 mg of 1.6 μm of gold microcarriers (Bio-Rad, Hercules, Calif.) and 100 μl of 0.05 M spermidine (Sigma, St, Louis, Mo.). Plasmid DNA (50 μg) and 1.0 M CaCl2 (100 μl) were added sequentially to the microcarriers while mixing by vortex. This mixture was allowed to precipitate at room temperature for 10 min. The microcarrier/DNA suspension was then centrifuged (10,000 rpm for 5 s) and washed three times in fresh absolute ethanol before resuspending in 3 ml of polyvinylpyrrolidone (0.1 mg/ml; Bio-Rad, Hercules, Calif.) in absolute ethanol. The solution was then loaded into tubing and allowed to settle for 4 min. The ethanol was gently removed, and the microcarrier/DNA suspension was evenly attached to the inside surface of the tubing by rotating the tube. The tube was then dried by 0.4 liters/min of flowing nitrogen gas. The dried tubing coated with microcarrier/DNA was then cut to 0.5-inch cartridges and stored in a capped dry bottle at 4□C. As a result, each cartridge contained 1 μg of plasmid DNA and 0.5 mg of gold. The DNA-coated gold particles (1 μg of DNA/bullet) were delivered to the shaved abdominal region of the mice using a helium-driven gene gun (Bio-Rad, Hercules, Calif.) with a discharge pressure of 400 p.s.i.

### Example 5

### Hydrodynamic Gene Transfer

Hydrodynamic transfection into mouse liver: The direct injections into the liver were done through either the portal vein or hepatic vein (via the inferior vena cava) under optimal conditions for expression as previously reported (Zhang et al., 1997). The optimal conditions entailed the injection of PDNA (plasmid DNA) in 1 ml of normal saline (0.9% NaCl) containing 15% mannitol (Sigma, St. Louis, Mo.) and heparin (2.5 units/ml; Lypho-Med, Chicago, Ill.). For the portal vein injections, outflow was blocked by occluding the hepatic vein and inferior vena cava. For the hepatic vein injections, outflow was blocked by occluding the portal vein, vena cava, and hepatic artery. The tail vein injections were done by injecting through a 27-gauge needle 10-250 micrograms of pDNA in 1-3 ml of Ringer's solutions (147 mM NaCl, 4 mM KCl, 1.13 mM CaCl2) over 7-120 sec.

### Example 6

### Transfection-Initiated Nanovector Transmission (TNT) by Hydrodynamic Injection into Mouse Liver

6.1 Plasmid pACE-GFP

Plasmid PACE-GFP contains the full sequence of the replication-competent amphotropic murine leukemia virus (MLV) vector construct, in which the cytomegalovirus (CMV) promoter has been used to replace the 5′ long terminal repeat (LTR) U3 region, and an encephalomyocarditis virus internal ribosome entry site (IRES)-green fluorescent protein (GFP) expression cassette is inserted between the env gene and 3′ LTR. The plasmid backbone contains the E. coli origin of replication and an ampicillin resistance gene. The complete sequence of pACE-GFP is given below, wherein

G=transcriptional start site (TSS) of viral mRNA. This represents the strart of the Moloney murine leukemia virus (MLV) sequences.

TSS is set as position 1 of MLV genomic sequence, so:

5′ long terminal repeat (LTR) R/U5 region=1 to 145

gag polyprotein sequence=621 to 2237

pol polyprotein sequence=2238 to 5837

Underlined=amphotropic MLV 4070A envelope coding sequence

3′ LTR=7816 to 8332

A=end of MLV sequence

Remaining sequences: plasmid backbone containing E. coli origin of replication and ampicillin resistance gene

TTTGAAAGACCCCACCCGTAGGTGGCAAGCTAGCTTAAGTAACGCCATTTTGCAAGGCATGGAAAAATAC
ATAACTGAGAATAGAGAAGTTCAGATCAAGGTCAGGAACAGATGGAACAGCTGAATATGGGCCAAACAGG
ATATCTGTGGTAAGCAGTTCCTGCCCCGGCTCAGGGCCAAGAACAGATGGAACAGCTGAATATGGGCCAA
ACAGGATATCTGTGGTAAGCAGTTCCTGCCCCGGCTCAGGGCCAAGAACAGATGGTCCCCAGATGCGGTC
CAGCCCTCAGCAGTTTCTAGAGAACCATCAGATGTTTCCAGGGTGCCCCAAGGACCTGAAATGACCCTGT
GCCTTATTTGAACTAACCAATCAGTTCGCTTCTCGCTTCTGTTCGCGCGCTTCTGCTCCCCGAGCTCAAT
AAAAGAGCCCACAACCCCTCACTCGGGGCGCCAGTCCTCCGATTGACTGAGTCGCCCGGGTACCCGTGTA
TCCAATAAACCCTCTTGCAGTTGCATCCGACTTGTGGTCTCGCTGTTCCTTGGGAGGGTCTCCTCTGAGT
GATTGACTACCCGTCAGCGGGGGTCTTTCATTTGGGGGCTCGTCCGGGATCGGGAGACCCCTGCCCAGGG
ACCACCGACCCACCACCGGGAGGTAAGCTGGCCAGCAACTTATCTGTGTCTGTCCGATTGTCTAGTGTCT
ATGACTGATTTTATGCGCCTGCGTCGGTACTAGTTAGCTAACTAGCTCTGTATCTGGCGGACCCGTGGTG
GAACTGACGAGTTCGGAACACCCGGCCGCAACCCTGGGAGACGTCCCAGGGACTTCGGGGGCCGTTTTTG
TGGCCCGACCTGAGTCCAAAAATCCCGATCGTTTTGGACTCTTTGGTGCACCCCCCTTAGAGGAGGGATA
TGTGGTTCTGGTAGGAGACGAGAACCTAAAACAGTTCCCGCCTCCGTCTGAATTTTTGCTTTCGGTTTGG
GACCGAAGCCGCGCCGCGCGTCTTGTCTGCTGCAGCATCGTTCTGTGTTGTCTCTGTCTGACTGTGTTTC
TGTATTTGTCTGAGAATATGGGCCAGACTGTTACCACTCCCTTAAGTTTGACCTTAGGTCACTGGAAAGA
TGTCGAGCGGATCGCTCACAACCAGTCGGTAGATGTCAAGAAGAGACGTTGGGTTACCTTCTGCTCTGCA
GAATGGCCAACCTTTAACGTCGGATGGCCGCGAGACGGCACCTTTAACCGAGACCTCATCACCCAGGTTA
AGATCAAGGTCTTTTCACCTGGCCCGCATGGACACCCAGACCAGGTCCCCTACATCGTGACCTGGGAAGC
CTTGGCTTTTGACCCCCCTCCCTGGGTCAAGCCCTTTGTACACCCTAAGCCTCCGCCTCCTCTTCCTCCA
TCCGCCCCGTCTCTCCCCCTTGAACCTCCTCGTTCGACCCCGCCTCGATCCTCCCTTTATCCAGCCCTCA
CTCCTTCTCTAGGCGCCAAACCTAAACCTCAAGTTCTTTCTGACAGTGGGGGGCCGCTCATCGACCTACT
TACAGAAGACCCCCCGCCTTATAGGGACCCAAGACCACCCCCTTCCGACAGGGACGGAAATGGTGGAGAA
GCGACCCCTGCGGGAGAGGCACCGGACCCCTCCCCAATGGCATCTCGCCTACGTGGGAGACGGGAGCCCC
CTGTGGCCGACTCCACTACCTCGCAGGCATTCCCCCTCCGCGCAGGAGGAAACGGACAGCTTCAATACTG
GCCGTTCTCCTCTTCTGACCTTTACAACTGGAAAAATAATAACCCTTCTTTTTCTGAAGATCCAGGTAAA
CTGACAGCTCTGATCGAGTCTGTTCTCATCACCCATCAGCCCACCTGGGACGACTGTCAGCAGCTGTTGG
GGACTCTGCTGACCGGAGAAGAAAAACAACGGGTGCTCTTAGAGGCTAGAAAGGCGGTGCGGGGCGATGA
TGGGCGCCCCACTCAACTGCCCAATGAAGTCGATGCCGCTTTTCCCCTCGAGCGCCCAGACTGGGATTAC
ACCACCCAGGCAGGTAGGAACCACCTAGTCCACTATCGCCAGTTGCTCCTAGCGGGTCTCCAAAACGCGG
GCAGAAGCCCCACCAATTTGGCCAAGGTAAAAGGAATAACACAAGGGCCCAATGAGTCTCCCTCGGCCTT
CCTAGAGAGACTTAAGGAAGCCTATCGCAGGTACACTCCTTATGACCCTGAGGACCCAGGGCAAGAAACT
AATGTGTCTATGTCTTTCATTTGGCAGTCTGCCCCAGACATTGGGAGAAAGTTAGAGAGGTTAGAAGATT
TAAAAAACAAGACGCTTGGAGATTTGGTTAGAGAGGCAGAAAAGATCTTTAATAAACGAGAAACCCCGGA
AGAAAGAGAGGAACGTATCAGGAGAGAAACAGAGGAAAAAGAAGAACGCCGTAGGACAGAGGATGAGCAG
AAAGAGAAAGAAAGAGATCGTAGGAGACATAGAGAGATGAGCAAGCTATTGGCCACTGTCGTTAGTGGAC
AGAAACAGGATAGACAGGGAGGAGAACGAAGGAGGTCCCAACTCGATCGCGACCAGTGTGCCTACTGCAA
AGAAAAGGGGCACTGGGCTAAAGATTGTCCCAAGAAACCACGAGGACCTCGGGGACCAAGACCCCAGACC
TCCCTCCTGACCCTAGATGACTAGGGAGGTCAGGGTCAGGAGCCCCCCCCTGAACCCAGGATAACCCTCA
AAGTCGGGGGGCAACCCGTCACCTTCCTGGTAGATACTGGGGCCCAACACTCCGTGCTGACCCAAAATCC
TGGACCCCTAAGTGATAAGTCTGCCTGGGTCCAAGGGGCTACTGGAGGAAAGCGGTATCGCTGGACCACG
GATCGCAAAGTACATCTAGCTACCGGTAAGGTCACCCACTCTTTCCTCCATGTACCAGACTGTCCCTATC
CTCTGTTAGGAAGAGATTTGCTGACTAAACTAAAAGCCCAAATCCACTTTGAGGGATCAGGAGCTCAGGT
TATGGGACCAATGGGGCAGCCCCTGCAAGTGTTGACCCTAAATATAGAAGATGAGCATCGGCTACATGAG
ACCTCAAAAGAGCCAGATGTTTCTCTAGGGTCCACATGGCTGTCTGATTTTCCTCAGGCCTGGGCGGAAA
CCGGGGGCATGGGACTGGCAGTTCGCCAAGCTCCTCTGATCATACCTCTGAAAGCAACCTCTACCCCCGT
GTCCATAAAACAATACCCCATGTCACAAGAAGCCAGACTGGGGATCAAGCCCCACATACAGAGACTGTTG
GACCAGGGAATACTGGTACCCTGCCAGTCCCCCTGGAACACGCCCCTGCTACCCGTTAAGAAACCAGGGA
CTAATGATTATAGGCCTGTCCAGGATCTGAGAGAAGTCAACAAGCGGGTGGAAGACATCCACCCCACCGT
GCCCAACCCTTACAACCTCTTGAGCGGGCTCCCACCGTCCCACCAGTGGTACACTGTGCTTGATTTAAAG
GATGCCTTTTTCTGCCTGAGACTCCACCCCACCAGTCAGCCTCTCTTCGCCTTTGAGTGGAGAGATCCAG
AGATGGGAATCTCAGGACAATTGACCTGGACCAGACTCCCACAGGGTTTCAAAAACAGTCCCACCCTGTT
TGATGAGGCACTGCACAGAGACCTAGCAGACTTCCGGATCCAGCACCCAGACTTGATCCTGCTACAGTAC
GTGGATGACTTACTGCTGGCCGCCACTTCTGAGCTAGACTGCCAACAAGGTACTCGGGCCCTGTTACAAA
CCCTAGGGAACCTCGGGTATCGGGCCTCGGCCAAGAAAGCCCAAATTTGCCAGAAACAGGTCAAGTATCT
GGGGTATCTTCTAAAAGAGGGTCAGAGATGGCTGACTGAGGCCAGAAAAGAGACTGTGATGGGGCAGCCT
ACTCCGAAGACCCCTCGACAACTAAGGGAGTTCCTAGGGACGGCAGGCTTCTGTCGCCTCTGGATCCCTG
GGTTTGCAGAAATGGCAGCCCCCTTGTACCCTCTCACCAAAACGGGGACTCTGTTTAATTGGGGCCCAGA
CCAACAAAAGGCCTATCAAGAAATCAAGCAAGCTCTTCTAACTGCCCCAGCCCTGGGGTTGCCAGATTTG
ACTAAGCCCTTTGAACTCTTTGTCGACGAGAAGCAGGGCTACGCCAAAGGTGTCCTAACGCAAAAACTGG
GACCTTGGCGTCGGCCGGTGGCCTACCTGTCCAAAAAGCTAGACCCAGTAGCAGCTGGGTGGCCCCCTTG
CCTACGGATGGTAGCAGCCATTGCCGTACTGACAAAGGATGCAGGCAAGCTAACCATGGGACAGCCACTA
GTCATTCTGGCCCCCCATGCAGTAGAGGCACTAGTCAAACAACCCCCCGACCGCTGGCTTTCCAACGCCC
GGATGACTCACTATCAGGCCTTGCTTTTGGACACGGACCGGGTCCAGTTCGGACCGGTGGTAGCCCTGAA
CCCGGCTACGCTGCTCCCACTGCCTGAGGAAGGGCTGCAACACAACTGCCTTGATATCCTGGCCGAAGCC
CACGGAACCCGACCCGACCTAACGGACCAGCCGCTCCCAGACGCCGACCACACCTGGTACACGGATGGAA
GCAGTCTCTTACAAGAGGGACAGCGTAAGGCGGGAGCTGCGGTGACCACCGAGACCGAGGTAATCTGGGC
TAAAGCCCTGCCAGCCGGGACATCCGCTCAGCGGGCTGAACTGATAGCACTCACCCAGGCCCTAAAGATG
GCAGAAGGTAAGAAGCTAAATGTTTATACTGATAGCCGTTATGCTTTTGCTACTGCCCATATCCATGGAG
AAATATACAGAAGGCGTGGGTTGCTCACATCAGAAGGCAAAGAGATCAAAAATAAAGACGAGATCTTGGC
CCTACTAAAAGCCCTCTTTCTGCCCAAAAGACTTAGCATAATCCATTGTCCAGGACATCAAAAGGGACAC
AGCGCCGAGGCTAGAGGCAACCGGATGGCTGACCAAGCGGCCCGAAAGGCAGCCATCACAGAGACTCCAG
ACACCTCTACCCTCCTCATAGAAAATTCATCACCCTACACCTCAGAACATTTTCATTACACAGTGACTGA
TATAAAGGACCTAACCAAGTTGGGGGCCATTTATGATAAAACAAAGAAGTATTGGGTCTACCAAGGAAAA
CCTGTGATGCCTGACCAGTTTACTTTTGAATTATTAGACTTTCTTCATCAGCTGACTCACCTCAGCTTCT
CAAAAATGAAGGCTCTCCTAGAGAGAAGCCACAGTCCCTACTACATGCTGAACCGGGATCGAACACTCAA
AAATATCACTGAGACCTGCAAAGCTTGTGCACAAGTCAACGCCAGCAAGTCTGCCGTTAAACAGGGAACT
AGGGTCCGCGGGCATCGGCCCGGCACTCATTGGGAGATCGATTTCACCGAGATAAAGCCCGGATTGTATG
GCTATAAATATCTTCTAGTTTTTATAGATACCTTTTCTGGCTGGATAGAAGCCTTCCCAACCAAGAAAGA
AACCGCCAAGGTCGTAACCAAGAAGCTACTAGAGGAGATCTTCCCCAGGTTCGGCATGCCTCAGGTATTG
GGAACTGACAATGGGCCTGCCTTCGTCTCCAAGGTGAGTCAGACAGTGGCCGATCTGTTGGGGATTGATT
GGAAATTACATTGTGCATACAGACCCCAAAGCTCAGGCCAGGTAGAAAGAATGAATAGAACCATCAAGGA
GACTTTAACTAAATTAACGCTTGCAACTGGCTCTAGAGACTGGGTGCTCCTACTCCCCTTAGCCCTGTAC
CGAGCCCGCAACACGCCGGGCCCCCATGGCCTCACCCCATATGAGATCTTATATGGGGCACCCCCGCCCC
TTGTAAACTTCCCTGACCCTGACATGACAAGAGTTACTAACAGCCCCTCTCTCCAAGCTCACTTACAGGC
TCTCTACTTAGTCCAGCACGAAGTCTGGAGACCTCTGGCGGCAGCCTACCAAGAACAACTGGACCGACCG
GTGGTACCTCACCCTTACCGAGTCGGCGACACAGTGTGGGTCCGCCGACACCAGACTAAGAACCTAGAAC
CTCGCTGGAAAGGACCTTACACAGTCCTGCTGACCACCCCCACCGCCCTCAAAGTAGACGGCATCGCAGC
TTGGATACACGCCGCCCACGTGAAGGCTGCCGACCCCGGGGGTGGACCATCCTCTAGACTGACATGGCGC
GTTCAACGCTCTCAAAACCCCCTCAAGATAAGATTAACCCGTGGAAGCCCTTAATAGTCATGGGAGTCCT
GTTAGGAGTAGGGATGGCAGAGAGCCCCCATCAGGTCTTTAATGTAACCTGGAGAGTCACCAACCTGATG
ACTGGGCGTACCGCCAATGCCACCTCCCTCCTGGGAACTGTACAAGATGCCTTCCCAAAATTATATTTTG
ATCTATGTGATCTGGTCGGAGAGGAGTGGGACCCTTCAGACCAGGAACCGTATGTCGGGTATGGCTGCAA
GTACCCCGCAGGGAGACAGCGGACCCGGACTTTTGACTTTTACGTGTGCCCTGGGCATACCGTAAAGTCG
GGGTGTGGGGGACCAGGAGAGGGCTACTGTGGTAAATGGGGGTGTGAAACCACCGGACAGGCTTACTGGA
AGCCCACATCATCGTGGGACCTAATCTCCCTTAAGCGCGGTAACACCCCCTGGGACACGGGATGCTCTAA
AGTTGCCTGTGGCCCCTGCTACGACCTCTCCAAAGTATCCAATTCCTTCCAAGGGGCTACTCGAGGGGGC
AGATGCAACCCTCTAGTCCTAGAATTCACTGATGCAGGAAAAAAGGCTAACTGGGACGGGCCCAAATCGT
GGGGACTGAGACTGTACCGGACAGGAACAGATCCTATTACCATGTTCTCCCTGACCCGGCAGGTCCTTAA
TGTGGGACCCCGAGTCCCCATAGGGCCCAACCCAGTATTACCCGACCAAAGACTCCCTTCCTCACCAATA
GAGATTGTACCGGCTCCACAGCCACCTAGCCCCCTCAATACCAGTTACCCCCCTTCCACTACCAGTACAC
CCTCAACCTCCCCTACAAGTCCAAGTGTCCCACAGCCACCCCCAGGAACTGGAGATAGACTACTAGCTCT
AGTCAAAGGAGCCTATCAGGCGCTTAACCTCACCAATCCCGACAAGACCCAAGAATGTTGGCTGTGCTTA
GTGTCGGGACCTCCTTATTACGAAGGAGTAGCGGTCGTGGGCACTTATACCAATCATTCCACCGCTCCGG
CCAACTGTACGGCCACTTCCCAACATAAGCTTACCCTATCTGAAGTGACAGGACAGGGCCTATGCATGGG
GGCAGTACCTAAAACTCACCAGGCCTTATGTAACACCACCCAAAGCGCCGGCTCAGGATCCTACTACCTT
GCAGCACCCGCCGGAACAATGTGGGCTTGCAGCACTGGATTGACTCCCTGCTTGTCCACCACGGTGCTCA
ATCTAACCACAGATTATTGTGTATTAGTTGAACTCTGGCCCAGAGTAATTTACCACTCCCCCGATTATAT
GTATGGTCAGCTTGAACAGCGTACCAAATATAAAAGAGAGCCAGTATCATTGACCCTGGCCCTTCTACTA
GGAGGATTAACCATGGGAGGGATTGCAGCTGGAATAGGGACGGGGACCACTGCCTTAATTAAAACCCAGC
AGTTTGAGCAGCTTCATGCCGCTATCCAGACAGACCTCAACGAAGTCGAAAAGTCAATTACCAACCTAGA
AAAGTCACTGACCTCGTTGTCTGAAGTAGTCCTACAGAACCGCAGAGGCCTAGATTTGCTATTCCTAAAG
GAGGGAGGTCTCTGCGCAGCCCTAAAAGAAGAATGTTGTTTTTATGCAGACCACACGGGGCTAGTGAGAG
ACAGCATGGCCAAATTAAGAGAAAGGCTTAATCAGAGACAAAAACTATTTGAGACAGGCCAAGGATGGTT
CGAAGGGCTGTTTAATAGATCCCCCTGGTTTACCACCTTAATCTCCACCATCATGGGACCTCTAATAGTA
CTCTTACTGATCTTACTCTTTGGACCTTGCATTCTCAATCGATTGGTCCAATTTGTTAAAGACAGGATCT
CAGTGGTCCAGGCTCTGGTTTTGACTCAGCAATATCACCAGCTAAAACCCATAGAGTACGAGCCATGAAC
GCGTTACTGGCCGAAGCCGCTTGGAATAAGGCCGGTGTGCGTTTGTCTATATGTTATTTTCCACCATATT
GCCGTCTTTTGGCAATGTGAGGGCCCGGAAACCTGGCCCTGTCTTCTTGACGAGCATTCCTAGGGGTCTT
TCCCCTCTCGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAGGTGCCTCTG
CGGCCAAAAGCCACGTGTATAAGATACACCTGCAAAGGCGGCACAACCCCAGTGCCACGTTGTGAGTTGG
ATAGTTGTGGAAAGAGTCAAATGGCTCTCCTCAAGCGTATTCAACAAGGGGCTGAAGGATGCCCAGAAGG
TACCCCATTGTATGGGATCTGATCTGGGGCCTCGGTGCACATGCTTTACATGTGTTTAGTCGAGGTTAAA
AAAACGTCTAGGCCCCCCGAACCACGGGGACGTGGTTTTCCTTTGAAAAACACGATTATAATATGGCCAG
CAAGGGCGAGGAGCTGTTCACCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCTGACCCTGAAGTTCATCTGCA
CCACCGGCAAGCTGCCCGTGCCCTGGCCCACCCTCGTGACCACCTTGACCTACGGCGTGCAGTGCTTCGC
CCGCTACCCCGACCACATGAAGCAGCACGACTTCTTCAAGTCCGCCATGCCCGAAGGCTACGTCCAGGAG
CGCACCATCTTCTTCAAGGACGACGGCAACTACAAGACCCGCGCCGAGGTGAAGTTCGAGGGCGACACCC
TGGTGAACCGCATCGAGCTGAAGGGCATCGACTTCAAGGAGGACGGCAACATCCTGGGGCACAAGCTGGA
GTACAACTACAACAGCCACAAGGTCTATATCACCGCCGACAAGCAGAAGAACGGCATCAAGGTGAACTTC
AAGACCCGCCACAACATCGAGGACGGCAGCGTGCAGCTCGCCGACCACTACCAGCAGAACACCCCCATCG
GCGACGGCCCCGTGCTGCTGCCCGACAACCACTACCTGAGCACCCAGTCCGCCCTGAGCAAAGACCCCAA
CGAGAAGCGCGATCACATGGTCCTGCTGGAGTTCGTGACCGCCGCCGGGATCACTCTCGGCATGGACGAG
CTGTACAAGTGAGCGGCCGCAGATAAAATAAAAGATTTTATTTAGTCTCCAGAAAAAGGGGGGAATGAAA
GACCCCACCTGTAGGTTTGGCAAGCTAGCTTAAGTAACGCCATTTTGCAAGGCATGGAAAAATACATAAC
TGAGAATAGAGAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGG
ATATCTGTGGTAAGCAGTTCCTGCCCCGGCTCAGGGCCAAGAACAGATGGTCCCCAGATGCGGTCCAGCC
CTCAGCAGTTTCTAGAGAACCATCAGATGTTTCCAGGGTGCCCCAAGGACCTGAAATGACCCTGTGCCTT
ATTTGAACTAACCAATCAGTTCGCTTCTCGCTTCTGTTCGCGCGCTTCTGCTCCCCGAGCTCAATAAAAG
AGCCCACAACCCCTCACTCGGGGCGCCAGTCCTCCGATTGACTGAGTCGCCCGGGTACCCGTGTATCCAA
TAAACCCTCTTGCAGTTGCATCCGACTTGTGGTCTCGCTGTTCCTTGGGAGGGTCTCCTCTGAGTGATTG
ACTACCCGTCAGCGGGGGTCTTTCATTACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGTGGCGCTTTC
TCAATGCTCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTGCAGGCATCGTGGTGTCACGCTCGTCG
TTTGGTATGGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAACACGGG
ATAATACCGCGCCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGCACATTT
CCCCGAAAAGTGCCACCTGACGTCTAAGAAACCATTATTATCATGACATTAACCTATAAAAATAGGCGTA
TCACGAGGCCCTTTCGTCTTCAAGAATTCATACCAGATCACCGAAAACTGTCCTCCAAATGTGTCCCCCT
CACACTCCCAAATTCGCGGGCTTCTGCTCTTAGACCACTCTACCCTATTCCCCACACTCACCGGAGCCAA
AGCCGCGGCCCTTCCGTTTCTTTGCT

The retrovirus nanovector produced after transfection of plasmid pACE-GFP is known to replicate with high efficiency in both human and murine colorectal cancer cell lines.

6.2 In Vitro Tests

FIG. 4 shows the replication curves of plasmid pACE-GFP-derived retrovirus vectors in the WiDr human colorectal cancer cell line and the CT26.WT murine colorectal cancer cell line, respectively, after inoculation at different doses. The curves in the upper panels show the percentage of GFP-positive cells as determined by flow cytometric analysis every three days following initial inoculation at multiplicities of infection (MOI) of 0.1 and 0.01 (i.e., one infectious nanovector per 10 cells or per 100 cells, respectively). The lower panels show representative images of GFP expression in the infected cells taken by fluorescence microscopy on the indicated days post-inoculation.

6.3 Establishment of a Murine Model for Metastasis of Colorectal Cancer to the Liver:

To demonstrate the use of hydrodynamic transfection as a method to deliver plasmid into tumors in vivo for initiation of replicating virus transmission, a mouse model of colorectal cancer metastasis to the liver was established by intrasplenic injection of tumor cells. Murine colon adenocarcinoma cell line CT26, originally derived from Balb/c mice, which can be obtained from the American Type Culture Collection (Manassas, Va., USA), was maintained in RPMI 1640 media containing 10% fetal bovine serum and 1% penicillin-streptomycin in a humidified atmosphere of 5% CO₂. To establish the tumor, after making a left subcostal incision under Isoflurane anesthesia, CT26 tumor cells (5×10e4 cells) in 200 μl PBS were injected into the spleen of 6-week-old female Balb/c mice through a 30-gauge needle. After hemostasis for 5 minutes, splenectomy was performed and the incision was closed with wound clips.

Of course, other suitable cell lines may be employed in this type of tumor model in the appropriate host (immunocompetent syngeneic hosts for tumors derived from the same strain and species, or athymic immunodeficient hosts for tumors derived from allogeneic strains or xenogeneic species).

6.4 In Vivo Transfection

Two weeks after tumor cell inoculation, 30 μg of the pACE-GFP plasmid is mixed with TransIT-QR (Quick Recovery) Hydrodynamic Delivery Solution (Mirus Bio Corp., Madison, Wis., USA), in a total volume of 0.1 ml per gram of body weight per mouse (e.g., 2.0 ml total volume per mouse, for mice with a body weight of 20 g). Hydrodynamic injections of this plasmid DNA solution are performed under optimal conditions for gene expression as previously reported (Zhang et al., 1997), which entail infusion of the total volume within 4 to 7 seconds at a constant rate through a 27-gauge needle placed in the tail vein.

Analysis of Gfp Expression after Transfection-Initiated Virus Replication:

At various time points after plasmid administration, livers are excised under sterile conditions and GFP fluorescence in the tumors is visualized using a Xenogen-IVIS cooled CCD optical imaging system (Xenogen IVIS, Alameda, Calif., USA). Composite images composed of gray-scale background photographic images of the isolated organs and color overlaid images of the emitted fluorescent light are generated with Living Image Software (Xenogen) and IGOR—PRO Image Analysis Software (Wave Metrics, Lake Oswego, Oreg., USA).

FIG. 5 shows representative composite images after optical imaging of GFP fluorescence from livers isolated 48 hours after hydrodynamic injection of plasmid pACE-GFP under the conditions described above right panel, pACE-GFP). Interestingly, hydrodynamic injection appears to result in preferential transfection of multifocal CT26 tumors compared to normal liver parenchyma (visualized as white masses embedded in the background of darker normal liver tissue), perhaps due to the larger and more “leaky” fenestrations present in newly formed tumor neovasculature. As a vehicle control, hydrodynamic injection of the TransIT-QR solution without addition of plasmid DNA was also performed in parallel; this transfection shows no detectable GFP fluorescence signal (left panel, control). The color scale at right shows the relative intensity of fluorescent signal flux in photons per second per cm².

FIG. 6 shows representative composite images after optical imaging of GFP fluorescence from livers isolated on Day 21 and Day 28, at which point direct expression of GFP from transfected plasmid should be completely extinguished; thus, all GFP fluorescence should be derived from the replicating retrovirus nanovector (middle and right panels, ACE-GFP Day 21 and Day 28, respectively). Increasing spread of GFP can be observed over time in multiple tumor masses due to transmission of the GFP transgene by the replicating virus vector.

The color scale at right showing the relative intensity of fluorescent signal flux in photons per second per cm² is 10-fold higher than in the previous figure; this demonstrates that not only the area but also the overall intensity of GFP transgene expression is increasing over time compared to the initial plasmid transfection. As a vehicle control, optical imaging of livers isolated after hydrodynamic injection of the TransIT-QR solution alone without addition of plasmid DNA was also performed as above; again, there is no detectable GFP fluorescence signal (left panel, control).

FIG. 7 shows the results of fluorescence-activated cell sorter (FACS) analysis of dispersed tumor cells harvested immediately after dissection at serial time points during pACE-GFP-derived virus nanovector replication in vivo. The largest hepatic tumor of each mouse was removed, digested with collagenase/dispase, and analyzed immediately by FACS (n=4 at each time point). The graph shows the percentage of GFP-positive cells (Y axis) detected in the tumor samples at each time point (X axis). Again, the results demonstrate that the GFP transgene is being effectively transmitted throughout the tumor masses over time by the replicating virus.

FIG. 8 shows the results from PCR analysis of pACE-GFP-derived replicating virus integration of the GFP transgene. Primers specific for the GFP transgene, whose sequence is not normally present in the mouse genome, were used for PCR amplification of genomic DNA isolated from murine CT26 tumors in the liver or from various normal tissues in tumor-bearing mice (as labeled) Untransduced tumors were also amplified as a negative control (identified as “Tumor (negative)”). The upper panel shows a standard curve for PCR amplification of the GFP sequence directly from the pACE-GFP plasmid mixed with genomic DNA at different copy numbers (as shown). The lower panel shows amplification of the endogenous mouse beta-actin gene sequence (500 bp band) with another set of specific primers as an internal control to demonstrate the integrity of the genomic DNA samples and the PCR procedure. The results show that a robust signal specific for GFP (700 bp band) could be amplified only in genomic DNA from tumors in which pACE-GFP-derived virus replication is occurring; in contrast, the initial plasmid DNA transfection itself is only transient and would never result in such a high level of integration into genomic DNA.

What is claimed is:

1. A plasmid encoding a replication-competent retrovirus for use in therapy.

2. The plasmid according to claim 1, wherein the replication-competent retrovirus comprises:
a retroviral GAG coding sequence;
a retroviral POL coding sequence;
a retroviral ENV coding sequence;
retroviral Long Terminal Repeat (LTR) sequences;
and optionally one or more of the following elements;
a heterologous coding sequence operably linked to a regulatory nucleic acid sequence; and
one or more targeting sequences for cell- or tissue-specific targeting of the retrovirus.

3. The plasmid according to claim 1, wherein the plasmid contains a therapeutic gene.

4. The plasmid according to claim 1, wherein the plasmid contains a coding sequence.

5. The plasmid according to claim 3, wherein the therapeutic gene is operably linked to a promoter and/or enhancer.

6. The plasmid according to claim 3, wherein the coding sequence is operably linked to a promoter and/or enhancer.

7. The plasmid according claim 1, wherein the replication competent retrovirus is a lytic virus.

8. The plasmid according to claim 7, wherein the lytic virus is an adenovirus.

9. The plasmid according to claim 1, wherein the tropism of the virus is enhanced or altered.

10. The plasmid according to claim 1, wherein the method used to deliver the plasmid to a subject in need of therapy is hydrodynamic transfection.

11. A formulation comprising a plasmid according to claim 1 together with a transfection agent.

12. A method of producing a plasmid encoding a replication-competent retrovirus for use in therapy, said method comprising:
(a) providing a vector comprising nucleic acid encoding a replication-competent retrovirus in a host cell;
(b) culturing said host cell; and
(c) recovering said plasmid,
wherein said replication-competent retrovirus comprises:
a retroviral GAG coding sequence;
a retroviral POL coding sequence;
a retroviral ENV coding sequence;
retroviral Long Terminal Repeat (LTR) sequences;
and optionally one or more of the following elements;
a heterologous coding sequence operably linked to a regulatory nucleic acid sequence; and
one or more targeting sequences for cell- or tissue-specific targeting of the retrovirus.

13. A method of treatment of a human or animal patient comprising the in vivo transfection of a cell of said patient with a plasmid coding for a replication-competent retrovirus.

14. The method as claimed in claim 13, wherein the viral genome incorporates a therapeutic gene or coding sequence suitable for the treatment of a condition which the patient is suffering from.

15. The method as claimed in claim 14, wherein said condition is a cell proliferative disease, an immunological disease, a neuronal disorder, an acquired infection or inflammation.

16. The method as claimed in claim 14, wherein said condition is a cancer, severe combined immunodeficiency disease, Parkinson's disease, Hepatitis C infection or diabetes.


SEQUENCE LISTING (1 sequence)

SEQ ID NO: 1 - DNA, 12136 bp, artificial sequence
  plasmid
tttgaaagaccccacccgtaggtggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatgaacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattataatatggccagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcataccagatcaccgaaaactgtcctccaaatgtgtccccctcacactcccaaattcgcgggcttctgctcttagaccactctaccctattccccacactcaccggagccaaagccgcggcccttccgtttctttgct